(19) United States
(12) Patent Application Publication
(10) Pub. No.: US 2011/0041193 A1
(43) Pub. Date: Feb. 17, 2011

(54) NON-HUMAN MAMMAL MODEL OF EPILEPSY

(76) Inventors: Shinichi Hirose, Fukuoka (JP); Sunao Kaneko, Aomori (JP)

Correspondence Address: WENDEROTH, LIND & PONACK, L.L.P., 1030 15th Street, N.W.,, Suite 400 East, Washington, DC, 20005-1503, US

(21) Appl. No.: 12/867,329

(22) PCT Filed: Feb. 10, 2009
(86) PCT No.: PCT/JP2009/052230

§ 371 Date: Oct. 20, 2010

(30) Foreign Application Priority Data
Feb. 12, 2008 (JP) 2008-031002

(51) Int. Cl. A01K 67/00; C12N 15/63; C07H 21/04; C12Q 1/68

(52) U.S. Cl. 800/9; 800/25; 536/23.5; 435/6

(57) ABSTRACT

The present invention provides a genuine epilepsy model animal as an improvement over conventional epilepsy model animals which are socalled seizures model animals mainly causing seizure attacks to be forcibly induced. Also provided is a method that allows for easy identification of the recombinants.

The model non-human mammalian animal for human epilepsy according to the present invention is a human epilepsy model non-human mammalian animal, such as a rat model, that has the same genetic defects as those in human epilepsy. The model non-human mammalian animal has a mutated gene obtained by introducing a genetic mutation to the non-human mammalian DNA of the α4 subunit (CHRNA4) or β2 subunit (CHRNB2) of the neuronal nicotinic acetylcholinergic receptor gene associated with human autosomal dominant nocturnal frontal lobe epilepsy, and by introducing a specific probe. The human epilepsy model non-human mammalian animal of the present invention allows for easy identification of the recombinants.

### TECHNICAL FIELD

The present invention relates to a non-human mammal model of epilepsy. More particularly, the present invention relates to a model non-human mammalian animal for human epilepsy that has genetic defects homologous to the genes associated with human autosomal dominant nocturnal frontal lobe epilepsy and that spontaneously develops epileptic seizures. The present invention also relates to mutated genes that allow for easy identification of genetically recombinant individuals, a method of construction thereof, and a method for identifying such genes.

### BACKGROUND ART

Epilepsy is a relatively common neurological disorder affecting about 2% of the population in Japan. However, the molecular cause of the disease has remained unclarified for many years because epilepsy is the collective term including many different forms of the disease. In recent years, genetic defects regarding epilepsy are gradually becoming clarified with the focus placed mostly on familial epilepsy.

Autosomal dominant nocturnal frontal lobe epilepsy as one form of familial epilepsy is characterized by nocturnal epileptic seizures. There is the report that this is caused by genetic mutations in the α4 and β2 subunit genes CHRNA4 and CHRNB2 associated with the neuronal acetylcholinergic receptor (Non-Patent Document 1). Three kinds of genetic mutations S284L, S280F, and 291-292insL have so far been reported in CHRNA4 (Non-Patent Documents 2, 3, 4, and 5). In CHRNB2, three kinds of genetic mutations V287L, V287M, and I312M have so far been reported (Non-Patent Documents 6, 7, and 8).

As a means for the development and advancement of the diagnostics and treatment of epilepsy, socalled “epilepsy model animals” have been used. Conventional epilepsy model animals are “seizure model animals” which make use of an electrical stimulus or a convulsant such as pentylenetetrazol. Transgenic non-human mammals which develop epilepsy-like seizures in response to the introduction of a sugar-binding antibody gene are disclosed (Patent Document 1). Non-human animals which lack a function of the μ3B gene on the chromosome and develop a tonic-clonic seizure or symptoms of epilepsy in response to a postural change are created (Patent Document 2). These conventional model animals can be used as model animals of seizure attacks, however they are not called genuine model animals for human epilepsy from the standpoint of molecular biology.

Previously, the inventors of the present invention have found that human autosomal dominant nocturnal frontal lobe epilepsy is caused by the substitution of Ser for Leu at position 284 of the α4 subunit (CHRNA4) of the neuronal nicotinic acetylcholinergic receptor gene (Non-Patent Document 9).

Based on this finding, the inventors of the present invention have created a genetically recombinant epilepsy model animal with a mutation introduced into the rat neuronal acetylcholinergic receptor gene CHRNA4 by genetic modifications (Patent Document 3).

The prior art techniques, however, require screening of large numbers of recombinants, because of the low probability of homologous recombination occurring in creating the genetically recombinant model animals. Further, the screening of recombinants requires sequencing of a part of the gene, adding time and cost to the procedure. Under these circumstances, demands have been made for the development of a method for distinguishing recombinants without sequencing the gene in order to save time and cost greatly.
[Non-Patent Document 1] Hirose, S., et al., Neurology 53:1749-1753, 1999
[Non-Patent Document 2] Steinlein, O. K., et al. A missense mutation in the neuronal nicotinic acetylcholine receptor alpha 4 subunit is associated with autosomal dominant nocturnal frontal lobe epilepsy. Nat Genet 11, 201-203 (1995).
[Non-Patent Document 3] Hirose, S., et al. A novel mutation of CHRNA4 responsible for autosomal dominant nocturnal frontal lobe epilepsy. Neurology 53, 1749-1753 (1999).
[Non-Patent Document 4] Steinlein, O. K., at al. Independent occurrence of the CHRNA4 Ser248Phe mutation in a Norwegian family with nocturnal frontal lobe epilepsy. Epilepsia 41, 529-535 (2000).
[Non-Patent Document 5] Steinlein, O. K., et al. An insertion mutation of the CHRNA4 gene in a family with autosomal dominant nocturnal frontal lobe epilepsy. Hum Mol Genet 6, 943-947 (1997).
[Non-Patent Document 6] De Fusco, M., et al. The nicotinic receptor b2 subunit is mutant in nocturnal frontal lobe epilepsy. Nat Genet 26, 275-276 (2000).
[Nan-Patent Document 7] Phillips, L A., et al. CHRNB2 is the second acetylcholine receptor subunit associated with autosomal dominant nocturnal frontal lobe epilepsy. Am J Hum Genet 68, 225-231 (2001).
[Non-Patent Document 8] Bertrand, D., et al. The CHRNB2 mutation I312M is associated with epilepsy and distinct memory deficits. Neurobiol Dis 20, 799-804 (2005).
[Non-Patent Document 9] Hirose, S., et al. A novel mutation of CHRNA4 responsible for autosomal dominant nocturnal frontal lobe epilepsy. Neurology 53, 1749-1753 (1999).
[Patent Document 1] JP-A-2006-141217
[Patent Document 2] Japanese Patent No. 3853136
[Patent Document 3] JP-A-2005-245361

### DISCLOSURE OF INVENTION

### Problems to be Solved by the Invention

In order to meet such demands, the inventors of the present invention have conducted extensive studies, and found that a model animal for epilepsy can be obtained from a non-human mammalian animal by introducing genetic mutations associated with the genetic defects in human CHRNA4 or CHRNB2 into the cDNA of the homologous gene Chrna4 or Chrnb2 of a non-human animal, respectively, and by introducing a probe to a part of the base sequence of the mutated cDNA, a probe being devised to have a base sequence that differs from this part of the base sequence, but codes for the same amino acid sequence. The inventors have also found that the non-human mammalian animal created this way allows an easy identification of the recombinant individuals, and enables the mRNA expressed from the recombinant gene to be easily detected by distinguishing it from the mRNA of the original homologous gene. It has further been found that the recombinant individuals created by introducing genetic mutations associated with the genetic defects in human CHRNA4 or CHRNB2 into the cDNA of the homologous gene Chrna4 or Chrnb2 of the non-human animal can easily be identified by devising the mutation introducing site and the mutation in such a manner that a new restriction site of a restriction enzyme can be created upon introduction of the mutation. The present invention was completed based on these findings.

It is accordingly an object of the present invention to provide a model non-human mammalian animal for human epilepsy, such as a model rat, which has the same genetic defects as those in human epilepsy. The model non-human mammalian animal includes a mutated gene obtained by introducing a genetic mutation into the non-human mammalian DNA of the α4 subunit (CHRNA4) or β2 subunit (CHRNB2) of the neuronal nicotinic acetylcholinergic receptor gene associated with human autosomal dominant nocturnal frontal lobe epilepsy, and by introducing a specific probe. The invention also has an object to provide a method for creating such model non-human mammalian animals for human epilepsy.

Another object of the present invention is to provide the mutated gene that includes the genetic mutation in the cDNA, and in which a part of the base sequence of the cDNA is replaced by a probe that has a base sequence different from this part of the base sequence, but codes for the same amino acid sequence. The invention also has an object to provide a method for creating such mutated genes.

In a preferred aspect, the present invention has an object to provide the mutated gene in which a new restriction site for a restriction enzyme is created by the introduction of the genetic mutation in the cDNA and to provide a method for introducing such mutations.

It is yet another object of the present invention to provide a method for the identification of a recombinant, which allows the recombinant individual to be easily identified with the restriction enzyme to be used for the restriction site of the restriction enzyme or with the restriction enzyme used to create the probe.

### Means for Solving the Problems

In order to achieve the foregoing objects, the present invention provides a model non-human mammalian animal for human epilepsy, wherein a mutated gene is created by introducing a genetic mutation to the gene Chrna4 or Chrnb2 of a non-human mammalian animal homologous to the α4 subunit (CHRNA4) or β2 subunit (CHRNB2) of the neuronal nicotinic acetylcholinergic receptor gene associated with human autosomal dominant nocturnal frontal lobe epilepsy, respectively, and wherein a part of the base sequence is replaced by introducing a probe devised to have a base sequence that differs from this part of the base sequence, but codes for the same amino acid sequence.

In a preferred aspect, the present invention provides a model non-human mammalian animal for human epilepsy, wherein a new restriction site for a restriction enzyme is caused to appear by the introduction of the mutation.

Another object of the present invention is to provide a method for creating the model non-human mammalian animal for human epilepsy. The method comprises preparing a mutated gene by introducing a genetic mutation into the cDNA of the gene Chrna4 or Chrnb2 of a non-human mammalian animal homologous to the α4 subunit CHRNA4 or β2 subunit CHRNB2 of the neuronal nicotinic acetylcholinergic receptor gene associated with human autosomal dominant nocturnal frontal lobe epilepsy, respectively, and by replacing a part of the base sequence of the cDNA by a probe that has a base sequence different from this part of the base sequence, yet codes for the same amino sequence, transferring the mutated gene to an expression vector, injecting the isolated DNA obtained by cutting it with an restriction enzyme into a fertilized egg, and transplanting the fertilized egg into a recipient female so as to create a recombinant.

The present invention also provides the mutated gene and a method for the production of the mutated gene, which includes one or more mutations introduced to its base sequence and in which a part of the cDNA base sequence is replaced by a probe that has a base sequence different from this part of the base sequence yet codes for the same amino sequence. In a preferred aspect of the invention, there is provided the mutated gene including a new restriction site for a restriction enzyme created by the introduction of the mutation, and a method for creating such mutated genes.

The present invention also provides a recombinant identification method which identifies the presence or absence of homologous recombination between the foreign gene as the mutated gene and the original gene of the host model non-human mammalian animal for human epilepsy, using the probe or the restriction enzyme for the restriction site.

### ADVANTAGE OF THE INVENTION

The model non-human mammalian animal for human epilepsy according to the present invention is highly advantageous in that it can be used as a genuine model animal for human epilepsy which naturally develops epileptic seizures, rather than a “seizure model animal” in which seizure attacks are forcibly induced with the use of an external stimulus or convulsants.

More specifically, the model non-human mammalian animal for human epilepsy of the present invention has the same genetic defects as those in human epilepsy so that the model non-human mammalian animal can be advantageously used as a genuine model animal for human epilepsy which naturally develops epileptic seizures as might occur in humans.

Another advantage of the model non-human mammalian animal for human epilepsy according to the present invention resides in an easy identification of a recombinant individual without sequencing the recombinants by the use of the restriction enzyme for the restriction site created by the introduction of the mutation or the use of the restriction enzyme for the probe introduced into the mutated gene. A further advantage of the present invention is to readily distinguish the mRNA expressing from the recombinant gene by PCR, in situ hybridization, etc. from the mRNA of the original Chrna4 or Chrnb2 of, for example, a rat.

### BRIEF DESCRIPTION OF DRAWINGS

FIG. 1 is a diagram illustrating enzyme sites at the Sma I/Bpul102 I site of CHRNA4, in which (A) shows the enzyme sites of wild-type CHRNA4, and (B) shows the enzyme sites of a probe.

FIG. 2 is a schematic diagram illustrating an expression vector constructed in Example 1.

FIG. 3 is a schematic diagram showing a structure of a PDGF promoter and rat Chrna4 at SnaB I/NaeI site.

FIG. 4 is a diagram illustrating enzyme sites at the Hinc II/Sma I site of CHRNB2, in which (A) shows the enzyme sites of wild-type CHRNB2, and (B) shows the enzyme sites of a probe.

FIG. 5 is a schematic diagram illustrating an expression vector constructed in Example 4.

FIG. 6 is a schematic diagram showing a structure of a PDGF promoter and rat Chrnb2 at SnaB I/Dra III Site.

FIG. 7 is a diagram representing the result of electroencephalography (EEG) for a transgenic rat (Chrnb2 V287L).

FIG. 8 is a magnified view of the portion A in FIG. 6.

FIG. 9 is a magnified view of the portion B in FIG. 6.

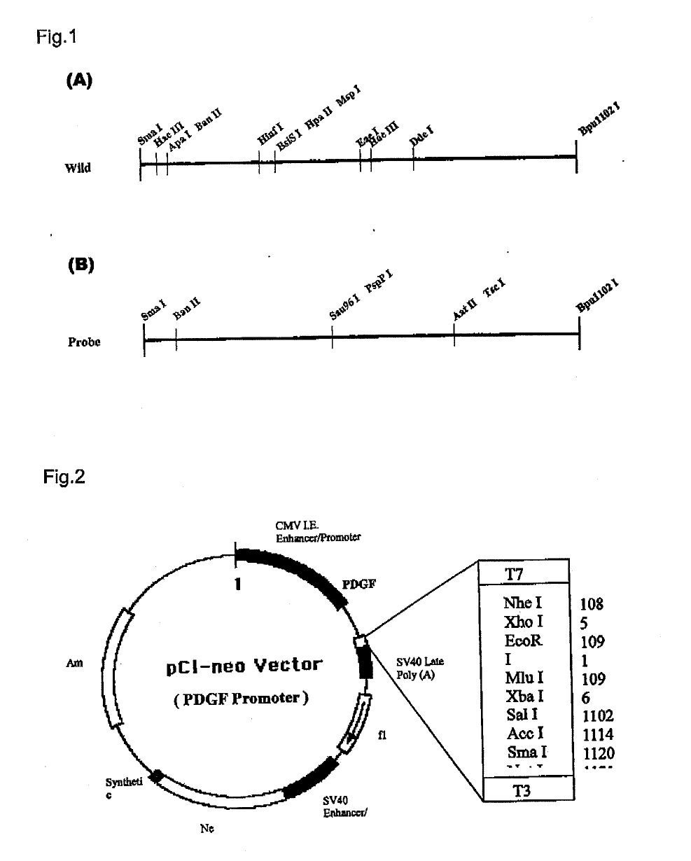

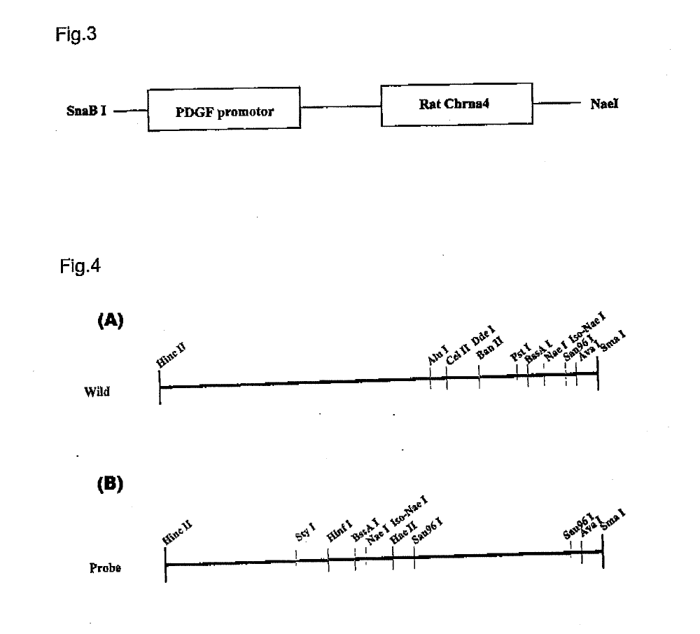

### BEST MODE FOR CARRYING OUT THE INVENTION

A model non-human mammalian animal for human epilepsy according to the present invention is a genetically recombinant model non-human mammalian animal for human epilepsy, in which a genetic mutation of the α4 subunit (CHRNA4) or the β2 subunit (CHRNB2) of a neuronal nicotinic acetylcholinergic receptor gene associated with human autosomal dominant nocturnal frontal lobe epilepsy that is one form of familial epilepsy and that is characterized by an occurrence of nocturnal epileptic seizures. Moreover, the genetically recombinant model non-human mammalian animal for human epilepsy possesses a mutated gene into which a probe is introduced, a probe being designed to allow a portion of the base sequence of the gene to have a base sequence that differs from the base sequence of the original gene yet codes for the amino acid sequence identical thereto. In addition, the function of the α4 subunit (CHRNA4) or the β2 subunit (CHRNB2) of the neuronal nicotinic acetylcholinergic receptor gene is deleted or lacks in the model animal.

As employed herein, the terms “non-human mammalian animals” refer to mammalian animals other than human beings, including mouse, rat, rabbit, dog, cat, swine, cattle, horse and so on. Among them, rats are preferred due to the fact that they have long been utilized for development of antiepileptics. It is further to be noted herein that the following description will be made mainly by taking rats as an example for brevity of explanation, but the present invention is not limted to rats.

At present, animal models for dieseases with genetic abnormality can be produced generally by genetic recombinant animal production techniques commonly used in the art. For the present invention, the human epilepsy model animals involved in human autosomal dominant nocturnal frontal lobe epilepsy that is one form of familial epilepsy and is characterized by nocturnal epileptic seizures can be produced by recombinant animal production techniques that have heretofore been conventionally used in the involved technical field.

At this end, a homologous gene of the α4 subunit (CHRNA4) or β2 subunit (CHRNB2) of the neuronal nicotinic acetylcholinergic receptor gene associated with human autosomal dominant nocturnal frontal lobe epilepsy is isolated from rats, etc. by techniques as used conventionally in the art, such as PCR cloning, or the like. The amino acid sequence of rat Chrna4 cDNA is deposited under NM_{—}000744 (NCBI) of human CHRNA4 cDNA database L31620 and the base sequence of rat Chrnb2 is deposited under NM_{—}019297 (NCBI).

The base sequence of the α4 subunit (Chrna4) of the rat neuronal nicotinic acetylcholinergic receptor gene (wild type) is illustrated as indicated immediately below. The underlined portion indicates the portion to be substituted for a probe in accordance with the present invention. The corresponding amino acid sequence is indicated as SEQ ID No. 30.

(SEQ ID No. 1)
ATGGCCAATTCGGGCACCGGGGCGCCGCCGCCGCTGCTGCTACTGCCGCTGCTGCTGCTCCTA
GGGACCGGCCTCTTGCCTGCTAGCAGCCACATAGAGACCCGGGCCCATGCGGAGGAGCGGCTC
CTGAAGAGACTCTTCTCCGGTTACAACAAGTGGTCTCGGCCAGTAGCCAATATCTCAGATGTG
GTCCTCGTCCGCTTTGGCTTGTCCATTGCTCAGCTCATTGACGTGGACGAGAAGAACCAGATG
ATGACAACCAACGTGTGGGTGAAGCAGGAGTGGCACGACTACAAGCTGCGCTGGGACCCTGGT
GACTACGAGAATGTCACCTCCATCCGCATCCCCTCTGAACTCATCTGGAGGCCTGACATCGTC
CTCTACAACAATGCGGATGGAGACTTTGCAGTCACCCACCTGACCAAGGCCCACCTGTTCTAT
GACGGAAGGGTGCAGTGGACACCCCCAGCCATCTATAAGAGCTCCTGCAGCATCGACGTCACC
TTCTTCCCCTTTGACCAGCAGAACTGTACCATGAAGTTTGGATCCTGGACCTACGACAAGGCC
AAGATTGACTTAGTGAGCATGCATAGCCGTGTGGACCAACTGGACTTCTGGGAAAGTGGGGAG
TGGGTCATCGTGGATGCTGTGGGCACCTACAACACCAGGAAGTACGAGTGCTGTGCCGAGATC
TATCCTGACATCACCTATGCCTTCATCATCCGACGGCTGCCGCTATTCTACACCATCAACCTC
ATCATCCCGTGCCTGCTCATCTCCTGTCTCACCGTGCTGGTCTTCTATCTGCCTTCAGAGTGT
GGCGAGAAGGTCACACTGTGCATCTCGGTGCTGCTTTCTCTCACCGTCTTCCTGCTGCTCATC
ACCGAGATCATCCCGTCCACCTCGCTGGTCATCCCGCTCATCGGCGAGTACCTCCTCTTCACC
ATGATCTTCGTCACCCTCTCCATCGTCATCACGGTCTTCGTGCTCAATGTGCACCACCGCTCG
CCACGCACACACACGATGCCCGCCTGGGTGCGTAGAGTCTTCCTGGACATCGTGCCTCGCCTC
CTCTTCATGAAGCGCCCCTCTGTGGTCAAAGACAACTGCCGGAGACTTATTGAGTCCATGCAC
AAGATGGCCAACGCCCCCCGCTTCTGGCCAGAGCCTGTGGGCGAGCCCGGCATCTTGAGTGAC
ATCTGCAACCAAGGTCTGTCACCTGCCCCAACTTTCTGCAACCCCACGGACACAGCAGTCGAG
ACCCAGCCTACGTGCAGGTCACCCCCCCTTGAGGTCCCTGACTTGAAGACATCAGAGGTTGAG
AAGGCCAGTCCCTGTCCATCGCCTGGCTCCTGTCCTCCACCCAAGAGCAGCAGTGGGGCTCCA
ATGCTCATCAAAGCCAGGTCCCTGAGTGTCCAGCATGTGCCCAGCTCCCAAGAAGCAGCAGAA
GATGGCATCCGCTGCCGGTCTCGGAGTATCCAGTACTGTGTTTCCCAAGATGGAGCTGCCTCC
CTGGCTGACAGCAAGCCCACCAGCTCCCCGACCTCCCTGAAGGCCCGTCCATCCCAGCTTCCC
GTGTCAGACCAGGCCTCTCCATGCAAATGCACATGCAAGGAACCATCTCCTGTGTCCCCAGTC
ACTGTGCTCAAGGCGGGAGGCACCAAAGCACCTCCCCAACACCTGCCCCTGTCACCAGCCCTG
ACACGGGCAGTAGAAGGCGTCCAGTACATTGCAGACCACCTCAAGGCAGAAGACACTGACTTC
TCGGTGAAGGAGGACTGGAAATACGTGGCCATGGTCATTGACCGAATCTTCCTCTGGATGTTC
ATCATTGTCTGCCTTCTGGGCACTGTGGGACTCTTCCTGCCTCCCTGGCTGGCTGCTTGCTGA

The base sequence of the β2 subunit (Chrnb2) of the rat neuronal nicotinic acetylcholinergic receptor gene (wild type) is illustrated as indicated immediately below. The underlined portion indicates the portion to be substituted for a probe in accordance with the present invention. The corresponding amino acid sequence is indicated as SEQ ID No. 31.

(SEQ ID No. 2)
ATGGCCGGGCACTCCAACTCAATGGCGCTGTTCAGCTTCAGCCTTCTTTGGCTGTGCTCAGGG
GTTTTGGGAACTGACACAGAGGAGCGGCTAGTGGAGCATCTCTTAGATCCCTCCCGCTATAAC
AAGCTGATTCGTCCAGCTACTAACGGCTCTGAGCTGGTGACTGTACAGCTCATGGTATCATTG
GCTCAGCTCATTAGTGTGCACGAGCGGGAGCAGATCATGACCACCAATGTCTGGCTGACCCAG
GAGTGGGAAGATTACCGCCTCACATGGAAGCCTGAGGACTTCGACAATATGAAGAAAGTCCGG
CTCCCTTCCAAACACATCTGGCTCCCAGATGTGGTTCTATACAACAATGCTGACGGCATGTAC
GAAGTCTCCTTCTATTCCAATGCTGTGGTCTCCTATGATGGCAGCATCTTTTGGCTACCACCT
GCCATCTACAAGAGTGCATGCAAGATTGAGGTGAAGCACTTCCCATTTGACCAGCAGAATTGC
ACCATGAAGTTTCGCTCATGGACCTACGACCGTACTGAGATTGACCTGGTGCTCAAAAGTGAT
GTGGCCAGTCTGGATGACTTCACACCCAGCGGGGAGTGGGACATCATCGCACTGCCAGGCCGA
CGCAACGAGAACCCAGACGACTCCACCTATGTGGACATCACCTATGACTTCATCATTCGTCGC
AAACCACTCTTCTACACTATCAACCTCATCATCCCCTGCGTACTCATCACCTCGCTGGCCATC
CTGGTCTTCTACCTGCCCTCAGACTGTGGTGAAAAGATGACACTTTGTATTTCTGTGCTGCTA
GCACTCACGGTGTTCCTGCTGCTCATCTCCAAGATTGTGCCTCCCACCTCCCTCGATGTACCG
CTGGTGGGCAAGTACCTCATGTTTACCATGGTGCTAGTCACCTTCTCCATCGTCACCAGCGTG
TGTGTGCTCAATGTGCACCACCGCTCGCCTACCACGCACACCATGGCCCCCTGGGTCAAGGTG
GTCTTCCTGGAGAAGCTGCCCACCCTGCTCTTCCTGCAGCAGCCACGCCACCGCTGTGCACGT
CAGCGTCTGCGCTTGAGGAGGCGCCAGCGAGAGCGTGAGGGCGCAGGCGCGCTTTTCTTCCGT
GAAGGTCCTGCGGCTGACCCATGTACCTGCTTTGTCAACCCTGCATCAGTGCAGGGCTTGGCT
GGGGCTTTCCGAGCTGAGCCCACTGCAGCCGGCCCGGGGCGCTCTGTGGGGCCATGCAGCTGT
GGCCTCCGGGAAGCAGTGGATGGCGTACGCTTCATTGCGGACCACATGCGAAGTGAGGATGAT
GACCAGAGTGTGAGGGAGGACTGGAAATACGTTGCCATGGTGATCGACCGCCTGTTCCTGTGG
ATCTTTGTCTTTGTCTGTGTCTTTGGGACCGTCGGCATGTTCCTGCAGCCTCTCTTCCAGAAC
TACACTGCCACTACCTTCCTCCACCCTGACCACTCAGCTCCCAGCTCCAAGTGA

The cDNA clones as prepared above may be subjected to mutation in accordance with mutation techniques known to the art. As the mutation techniques to be used for the present invention, there may be used conventional ones as have been frequently used in the art, such as site-directed mutagenesis. The site-directed mutagenesis allows an optional mutation to be inserted site-specifically into an optional site of a cDNA. The mutation to be site-specifically inserted into the optional site of the cDNA is not limited to a specific one and may include modifications, for example, such as deletion, defect, substitution, and addition.

Therefore, the present invention enables a specific mutation to be inserted into an isolated rat Chrna4 or Chrnb2 using the site-directed mutagenesis, resulting in the rat homologous gene Chrna4 or Chrnb2 with the mutation inserted therein, respectively, which possesses a base sequence coding for a protein having functions associated with the onset of nocturnal epileptic seizures derived from human autosomal dominant nocturnal frontal lobe epilepsy.

As used herein, the term “homologous gene” or the terms relating thereto refers to a gene which has a base sequence equivalent of that of a gene derived from an animal of a different species considered as having a common ancestor and which encodes a protein having equivalent functions.

In accordance with the present invention, the cDNA of the subunits of the rat neuronal nicotinic acetylcholinergic receptor gene may be prepared in the manner as will be described hereinafter.

More specifically, a set consisting of two kinds of primers, for example, a forward primer and a reverse primer, each having the following base sequence, is designed and prepared on the basis of the cDNA sequence of the known rat Chrna4 or Chrnb2 using the rat cDNA clone, respectively, as a template.

The base sequences of the forward primer (40mer) (SEQ ID No. 3) and the reverse primer (35mer) (SEQ ID No. 4), each prepared, respectively, on the basis of the cDNA sequence of the rat Chrna4, are indicated as below:

SEQ ID No. 3:
AGATCTCGCGAAGCTTCACCATGGCCAATTCGGGCACCGG
SEQ ID No. 4:
AGATCTAGATCAGCAAGCAGCCAGCCAGGGAGGCAGGA

The base sequences of the forward primer (41mer) (SEQ ID No. 5) and the reverse primer (40mer) (SEQ ID No. 6), each prepared, respectively, on the basis of the cDNA sequence of the rat Chrnb2, are indicated as below:

SEQ ID No. 5:
AGATCTCGCGACATGGCCGGGCACTCCAACTCAATGGCGCT
SEQ ID No. 6:
ATCGATGGATCCTCACTTGGAGCTGGGAGCTGAGTGGTCA

The PCR is performed using these primers and the resulting PCR products are sub-cloned in an appropriate vector. The resulting clones are then sequenced to confirm the base sequences of the Chrna4 and Chrnb2 cDNAs.

Thereafter, a mutation is inserted into an optional site of the resulting cDNA clones as obtained above. The mutation may be performed using various mutation techniques known to the art. An optional mutation may be introduced into an optional site using known mutation techniques in particular including site-directed mutagenesis.

As previously described, three mutations, i.e., S280F, S284L, and 291-292insL are reported as gene abnormality of the CHRNA4 gene. Three mutations, i.e., V287L, V287M, and I312M are reported as genetic mutations of the CHRNB2 gene.

Therefore, the present invention allows a mutation of a species-specific homologous gene corresponding to, for example, the mutations S280F, S284L or 291-292insL of the rat CHRNA4 gene or the mutations V287L, V287M or I312M of the rat CHRNB2 gene in the rat Chrna4 (wild type) (SEQ ID NO.1) and the rat Chrnb2 (wild type) (SEQ ID NO.2), respectively, isolated by the PCR cloning as performed above, to be performed by the above mutation techniques.

Thus, the mutation may be performed to introduce a specific mutation into a specific mutation site with a commercially available kit, using appropriate sense primers and anti-sense primers. The sense primer and anti-sense primer to be used for the mutation may be in the form of generally 20mer to 40mer, preferably 25mer to 35mer. In this case, it is preferred to design the mutation site and perform the mutation so as to allow a restriction site of a restriction enzyme to appear on a mutation site.

More specifically, for instance, the mutation S282F (i.e., S280F in humans) may be introduced in the wild-type rat Chrna4 using a sense primer (29mer) and an anti-sense primer (29mer) as indicated below to replace cytosine (C) at cDNA base sequence position 845 by thymine (T) (c.845C>T) and guanidine (G) at cDNA base sequence position 846 by cytosine (C) (c.846G>C). This mutation allows the amino acid residue Ser homologous to position 282 of CHRNA4 to be replaced by Phe.

The base sequences of the sense primer (SEQ ID No. 7) and the anti-sense primer (SEQ ID No. 8) to be used herein are indicated as follows:

SEQ ID No. 7:
CACACTGTGCATCTTCGTGCTGCTTTCTC
SEQ ID No. 8:
GAGAAAGCAGCACGAAGATGCACAGTGTG

The base sequence of the resulting mutated rat cDNA is indicated as follows;

(SEQ ID No. 9)
ATGGCCAATTCGGGCACCGGGGCGCCGCCGCCGCTGCTGCTACTGCCGCTGCTGCTGCTCCTA
GGGACCGGCCTCTTGCCTGCTAGCAGCCACATAGAGACCCGGCCCATGCGGAGGAGCGGCTCC
TGAAGAGACTCTTCTCCGGTTACAACAAGTGGTCTCGGCCAGTAGCCAATATCTCAGATGTGG
TCCTCGTCCGCTTTGGCTTGTCCATTGCTCAGCTCATTGACGTGGACGAGAAGAACCAGATGA
TGACAACCAACGTGTGGGTGAAGCAGGAGTGGCACGACTACAAGCTGCGCTGGGACCCTGGTG
ACTACGAGAATGTCACCTCCATCCGCATCCCCTCTGAACTCATCTGGAGGCCTGACATCGTCC
TCTACAACAATGCGGATGGAGACTTTGCAGTCACCCACCTGACCAAGGCCCACCTGTTCTATG
ACGGAAGGGTGCAGTGGACACCCCCAGCCATCTATAAGAGCTCCTGCAGCATCGACGTCACCT
TCTTCCCCTTTGACCAGCAGAACTGTACCATGAAGTTTGGATCCTGGACCTACGACAAGGCCA
AGATTGACTTAGTGAGCATGCATAGCCGTGTGGACCAACTGGACTTCTGGGAAAGTGGGGAGT
GGGTCATCGTGGATGCTGTGGGCACCTACAACACCAGGAAGTACGAGTGCTGTGCCGAGATCT
ATCCTGACATCACCTATGCCTTCATCATCCGACGGCTGCCGCTATTCTACACCATCAACCTCA
TCATCCCGTGCCTGCTCATCTCCTGTCTCACCGTGCTGGTCTTCTATCTGCCTTCAGAGTGTG
GCGAGAAGGTCACACTGTGCATCTTCGTGCTGCTTTCTCTCACCGTCTTCCTGCTGCTCATCA
CCGAGATCATCCCGTCCACCTCGCTGGTCATCCCGCTCATCGGCGAGTACCTCCTCTTCACCA
TGATCTTCGTCACCCTCTCCATCGTCATCACGGTCTTCGTGCTCAATGTGCACCACCGCTCGC
CACGCACACACACGATGCCCGCCTGGGTGCGTAGAGTCTTCCTGGACATCGTGCCTCGCCTCC
TCTTCATGAAGCGCCCCTCTGTGGTCAAAGACAACTGCCGGAGACTTATTGAGTCCATGCACA
AGATGGCCAACGCCCCCCGCTTCTGGCCAGAGCCTGTGGGCGAGCCCGGCATCTTGAGTGACA
TCTGCAACCAAGGTCTGTCACCTGCCCCAACTTTCTGCAACCCCACGGACACAGCAGTCGAGA
CCCAGCCTACGTGCAGGTCACCCCCCCTTGAGGTCCCTGACTTGAAGACATCAGAGGTTGAGA
AGGCCAGTCCCTGTCCATCGCCTGGCTCCTGTCCTCCACCCAAGAGCAGCAGTGGGGCTCCAA
TGCTCATCAAAGCCAGGTCCCTGAGTGTCCAGCATGTGCCCAGCTCCCAAGAAGCAGCAGAAG
ATGGCATCCGCTGCCGGTCTCGGAGTATCCAGTACTGTGTTTCCCAAGATGGAGCTGCCTCCC
TGGCTGACAGCAAGCCCACCAGCTCCCCGACCTCCCTGAAGGCCCGTCCATCCCAGCTTCCCG
TGTCAGACCAGGCCTCTCCATGCAAATGCACATGCAAGGAACCATCTCCTGTGTCCCCAGTCA
CTGTGCTCAAGGCGGGAGGCACCAAAGCACCTCCCCAACACCTGCCCCTGTCACCAGCCCTGA
CACGGGCAGTAGAAGGCGTCCAGTACATTGCAGACCACCTCAAGGCAGAAGACACTGACTTCT
CGGTGAAGGAGGACTGGAAATACGTGGCCATGGTCATTGACCGAATCTTCCTCTGGATGTTCA
TCATTGTCTGCCTTCTGGGCACTGTGGGACTCTTCCTGCCTCCCTGGCTGGCTGCTTGCTGA

Similarly, the mutation of S286L (i.e., S284L in humans) may be introduced into the wild-type rat Chrna4 using a sense primer (29mer) and an anti-sense primer (29mer) as indicated below to replace tyrosine (T) at base sequence position 856 of cDNA by cysteine (C) (c.856T>C) and cysteine (C) at base sequence position 857 thereof by tyrosine (T) (c.857C>T). This mutation results in replacement of the amino acid residue Ser homologous to position 286 of CHRNA4 by the amino acid residue Leu.

The base sequences of the sense primer (SEQ ID No. 10) and the anti-sense primer (SEQ ID No. 11) to be used herein are indicated as follows:

SEQ ID No. 10:
CGGTGCTGCTTCTTCTCACCGTCTTCCTG
SEQ ID No. 11:
CAGGAAGACGGTGAGAAGAAGCAGCACCG

The base sequence of the resulting mutated rat cDNA is indicated as follows:

(SEQ ID No. 12)
ATGGCCAATTCGGGCACCGGGGCGCCGCCGCCGCTGCTGCTACTGCCGCTGCTGCTGCTCCTA
GGGACCGGCCTCTTGCCTGCTAGCAGCCACATAGAGACCCGGGCCCATGCGGAGGAGCGGCTC
CTGAAGAGACTCTTCTCCGGTTACAACAAGTGGTCTCGGCCAGTAGCCAATATCTCAGATGTG
GTCCTCGTCCGCTTTGGCTTGTCCATTGCTCAGCTCATTGACGTGGACGAGAAGAACCAGATG
ATGACAACCAACGTGTGGGTGAAGCAGGAGTGGCACGACTACAAGCTGCGCTGGGACCCTGGT
GACTACGAGAATGTCACCTCCATCCGCATCCCCTCTGAACTCATCTGGAGGCCTGACATCGTC
CTCTACAACAATGCGGATGGAGACTTTGCAGTCACCCACCTGACCAAGGCCCACCTGTTCTAT
GACGGAAGGGTGCAGTGGACACCCCCAGCCATCTATAAGAGCTCCTGCAGCATCGACGTCACC
TTCTTCCCCTTTGACCAGCAGAACTGTACCATGAAGTTTGGATCCTGGACCTACGACAAGGCC
AAGATTGACTTAGTGAGCATGCATAGCCGTGTGGACCAACTGGACTTCTGGGAAAGTGGGGAG
TGGGTCATCGTGGATGCTGTGGGCACCTACAACACCAGGAAGTACGAGTGCTGTGCCGAGATC
TATCCTGACATCACCTATGCCTTCATCATCCGACGGCTGCCGCTATTCTACACCATCAACCTC
ATCATCCCGTGCCTGCTCATCTCCTGTCTCACCGTGCTGGTCTTCTATCTGCCTTCAGAGTGT
GGCGAGAAGGTCACACTGTGCATCTCGGTGCTGCTTCTTCTCACCGTCTTCCTGCTGCTCATC
ACCGAGATCATCCCGTCCACCTCGCTGGTCATCCCGCTCATCGGCGAGTACCTCCTCTTCACC
ATGATCTTCGTCACCCTCTCCATCGTCATCACGGTCTTCGTGCTCAATGTGCACCACCGCTCG
CCACGCACACACACGATGCCCGCCTGGGTGCGTAGAGTCTTCCTGGACATCGTGCCTCGCCTC
CTCTTCATGAAGCGCCCCTCTGTGGTCAAAGACAACTGCCGGAGACTTATTGAGTCCATGCAC
AAGATGGCCAACGCCCCCCGCTTCTGGCCAGAGCCTGTGGGCGAGCCCGGCATCTTGAGTGAC
ATCTGCAACCAAGGTCTGTCACCTGCCCCAACTTTCTGCAACCCCACGGACACAGCAGTCGAG
ACCCAGCCTACGTGCAGGTCACCCCCCCTTGAGGTCCCTGACTTGAAGACATCAGAGGTTGAG
AAGGCCAGTCCCTGTCCATCGCCTGGCTCCTGTCCTCCACCCAAGAGCAGCAGTGGGGCTCCA
ATGCTCATCAAAGCCAGGTCCCTGAGTGTCCAGCATGTGCCCAGCTCCCAAGAAGCAGCAGAA
GATGGCATCCGCTGCCGGTCTCGGAGTATCCAGTACTGTGTTTCCCAAGATGGAGCTGCCTCC
CTGGCTGACAGCAAGCCCACCAGCTCCCCGACCTCCCTGAAGGCCCGTCCATCCCAGCTTCCC
GTGTCAGACCAGGCCTCTCCATGCAAATGCACATGCAAGGAACCATCTCCTGTGTCCCCAGTC
ACTGTGCTCAAGGCGGGAGGCACCAAAGCACCTCCCCAACACCTGCCCCTGTCACCAGCCCTG
ACACGGGCAGTAGAAGGCGTCCAGTACATTGCAGACCACCTCAAGGCAGAAGACACTGACTTC
TCGGTGAAGGAGGACTGGAAATACGTGGCCATGGTCATTGACCGAATCTTCCTCTGGATGTTC
ATCATTGTCTGCCTTCTGGGCACTGTGGGACTCTTCCTGCCTCCCTGGCTGGCTGCTTGCTGA

In a manner similar to the above mutation, the mutated bases GCT (c.878-879insGCT) may be inserted in between the bases at positions 878 and 879 of the wild-type rat Chrna4 cDNA, thereby allowing an insertion of the amino acid residue leucine (Leu) to be inserted in between the amino acid residue leucine (Leu) homologous to the amino acid at position 293 (i.e., 291 in humans) of CHRNA4 and the amino acid residue isoleucine (Ile) homologous to the amino acid at position 294 (i.e., 292 in humans) thereof using a sense primer (30mer) and an anti-sense primer (30mer) as indicated below.

The base sequences of the sense primer (SEQ ID No. 13) and the anti-sense primer (SEQ ID No. 14) are indicated as follows:

SEQ ID No. 13:
GTCTTCCTGCTGCTGCTCATCACCGAGATC
SEQ ID No. 14:
GATCTCGGTGATGAGCAGCAGCAGGAAGAC

The base sequence of the cDNA resulting by the above mutation is indicated as follows:

(SEQ ID No. 15)
ATGGCCAATTCGGGCACCGGGGCGCCGCCGCCGCTGCTGCTACTGCCGCTGCTGCTGCTCCTA
GGGACCGGCCTCTTGCCTGCTAGCAGCCACATAGAGACCCGGGCCCATGCGGAGGAGCGGCTC
CTGAAGAGACTCTTCTCCGGTTACAACAAGTGGTCTCGGCCAGTACCCAATATCTCAGATGTG
GTCCTCGTCCGCTTTGGCTTGTCCATTGCTCAGCTCATTGACGTGGACGAGAAGAACCAGATG
ATGACAACCAACGTGTGGGTGAAGCAGGAGTGGCACGACTACAAGCTGCGCTGGGACCCTGGT
GACTACGAGAATGTCACCTCCATCCGCATCCCCTCTGAACTCATCTGGAGGCCTGACATCGTC
CTCTACAACAATGCGGATGGAGACTTTGCAGTCACCCACCTGACCAAGGCCCACCTGTTCTAT
GACGGAAGGGTGCAGTGGACACCCCCAGCCATCTATAAGAGCTCCTGCAGCATCGACGTCACC
TTCTTCCCCTTTGACCAGCAGAACTGTACCATGAAGTTTGGATCCTGGACCTACGACAAGGCC
AAGATTGACTTAGTGAGCATGCATAGCCGTGTGGACCAACTGGACTTCTGGGAAAGTGGGGAG
TGGGTCATCGTGGATGCTGTGGGCACCTACAACACCAGGAAGTACGAGTGCTGTGCCGAGATC
TATCCTGACATCACCTATGCCTTCATCATCCGACGGCTGCCGCTATTCTACACCATCAACCTC
ATCATCCCGTGCCTGCTCATCTCCTGTCTCACCGTGCTGGTCTTCTATCTGCCTTCAGAGTGT
GGCGAGAAGGTCACACTGTGCATCTCGGTGCTGCTTTCTCTCACCGTCTTCCTGCTGCTGCTA
ATCACCGAGATCATCCCGTCCACCTCGCTGGTCATCCCGCTCATCGGCGAGTACCTCCTCTTC
ACCATGATCTTCGTCACCCTCTCCATCGTCATCACGGTCTTCGTGCTCAATGTGCACCACCGC
TCGCCACGCACACACACGATGCCCGCCTGGGTGCGTAGAGTCTTCCTGGACATCGTGCCTCGC
CTCCTCTTCATGAAGCGCCCCTCTGTGGTCAAAGACAACTGCCGGAGACTTATTGAGTCCATG
CACAAGATGGCCAACGCCCCCCGCTTCTGGCCAGAGCCTGTGGGCGAGCCCGGCATCTTGAGT
GACATCTGCAACCAAGGTCTGTCACCTGCCCCAACTTTCTGCAACCCCACGGACACAGCAGTC
GAGACCCAGCCTACGTGCAGGTCACCCCCCCTTGAGGTCCCTGACTTGAAGACATCAGAGGTT
GAGAAGGCCAGTCCCTGTCCATCGCCTGGCTCCTGTCCTCCACCCAAGAGCAGCAGTGGGGCT
CCAATGCTCATCAAAGCCAGGTCCCTGAGTGTCCAGCATGTGCCCAGCTCCCAAGAAGCAGCA
GAAGATGGCATCCGCTGCCGGTCTCGGAGTATCCAGTACTGTGTTTCCCAAGATGGAGCTGCC
TCCCTGGCTGACAGCAAGCCCACCAGCTCCCCGACCTCCCTGAAGGCCCGTCCATCCCAGCTT
CCCGTGTCAGACCAGGCCTCTCCATGCAAATGCACATGCAAGGAACCATCTCCTGTGTCCCCA
GTCACTGTGCTCAAGGCGGGAGGCACCAAAGCACCTCCCCAACACCTGCCCCTGTCACCAGCC
CTGACACGGGCAGTAGAAGGCGTCCAGTACATTGCAGACCACCTCAAGGCAGAAGACACTGAC
TTCTCGGTGAAGGAGGACTGGAAATACGTGGCCATGGTCATTGACCGAATCTTCCTCTGGATG
TTCATCATTGTCTGCCTTCTGGGCACTGTGGGACTCTTCCTGCCTCCCTGGCTGGCTGCTTGC
TGA

In substantially the same manner as in the case of the above c.878-879insGCT, the bases TTA (c.879-880insTTA) may be inserted in between the bases at positions 879 and 880 of the wild-type rat Chrna4 cDNA, resulting in an insertion of the amino acid residue leucine (Leu) in between the amino acid residue leucine (Leu) homologous to the amino acid at position 293 (i.e., 291 in humans) of CHRNA4 and the amino acid residue isoleucine (Ile) homologous to the amino acid at position 294 (i.e., 292 in humans) thereof.

(SEQ ID No. 16)
ATGGCCAATTCGGGCACCGGGGCGCCGCCGCCGCTGCTGCTACTGCCGCTGCTGCTGCTCCTA
GGGACCGGCCTCTTGCCTGCTAGCAGCCACATAGAGACCCGGGCCCATGCGGAGGAGCGGCTC
CTGAAGAGACTCTTCTCCGGTTACAACAAGTGGTCTCGGCCAGTAGCCAATATCTCAGATGTG
GTCCTCGTCCGCTTTGGCTTGTCCATTGCTCAGCTCATTGACGTGGACGAGAAGAACCAGATG
ATGACAACCAACGTGTGGGTGAAGCAGGAGTGGCACGACTACAAGCTGCGCTGGGACCCTGGT
GACTACGAGAATGTCACCTCCATCCGCATCCCCTCTGAACTCATCTGGAGGCCTGACATCGTC
CTCTACAACAATGCGGATGGAGACTTTGCAGTCACCCACCTGACCAAGGCCCACCTGTTCTAT
GACGGAAGGGTGCAGTGGACACCCCCAGCCATCTATAAGAGCTCCTGCAGCATCGACGTCACC
TTCTTCCCCTTTGACCAGCAGAACTGTACCATGAAGTTTGGATCCTGGACCTACGACAAGGCC
AAGATTGACTTAGTGAGCATGCATAGCCGTGTGGACCAACTGGACTTCTGGGAAAGTGGGGAG
TGGGTCATCGTGGATGCTGTGGGCACCTACAACACCAGGAAGTACGAGTGCTGTGCCGAGATC
TATCCTGACATCACCTATGCCTTCATCATCCGACGGCTGCCGCTATTCTACACCATCAACCTC
ATCATCCCGTGCCTGCTCATCTCCTGTCTCACCGTGCTGGTCTTCTATCTGCCTTCAGAGTGT
GGCGAGAAGGTCACACTGTGCATCTCGGTGCTGCTTTCTCTCACCGTCTTCCTGCTGCTCTTA
ATCACCGAGATCATCCCGTCCACCTCGCTGGTCATCCCGCTCATCGGCGAGTACCTCCTCTTC
ACCATGATCTTCGTCACCCTCTCCATCGTCATCACGGTCTTCGTGCTCAATGTGCACCACCGC
TCGCCACGCACACACACGATGCCCGCCTGGGTGCGTAGAGTCTTCCTGGACATCGTGCCTCGC
CTCCTCTTCATGAAGCGCCCCTCTGTGGTCAAAGACAACTGCCGGAGACTTATTGAGTCCATG
CACAAGATGGCCAACGCCCCCCGCTTCTGGCCAGAGCCTGTGGGCGAGCCCGGCATCTTGAGT
GACATCTGCAACCAAGGTCTGTCACCTGCCCCAACTTTCTGCAACCCCACGGACACAGCAGTC
GAGACCCAGCCTACGTGCAGGTCACCCCCCCTTGAGGTCCCTGACTTGAAGACATCAGAGGTT
GAGAAGGCCAGTCCCTGTCCATCGCCTGGCTCCTGTCCTCCACCCAAGAGCAGCAGTGGGGCT
CCAATGCTCATCAAAGCCAGGTCCCTGAGTGTCCAGCATGTGCCCAGCTCCCAAGAAGCAGCA
GAAGATGGCATCCGCTGCCGGTCTCGGAGTATCCAGTACTGTGTTTCCCAAGATGGAGCTGCC
TCCCTGGCTGACAGCAAGCCCACCAGCTCCCCGACCTCCCTGAAGGCCCGTCCATCCCAGCTT
CCCGTGTCAGACCAGGCCTCTCCATGCAAATGCACATGCAAGGAACCATCTCCTGTGTCCCCA
GTCACTGTGCTCAAGGCGGGAGGCACCAAAGCACCTCCCCAACACCTGCCCCTGTCACCAGCC
CTGACACGGGCAGTAGAAGGCGTCCAGTACATTGCAGACCACCTCAAGGCAGAAGACACTGAC
TTCTCGGTGAAGGAGGACTGGAAATACGTGGCCATGGTCATTGACCGAATCTTCCTCTGGATG
TTCATCATTGTCTGCCTTCTGGGCACTGTGGGACTCTTCCTGCCTCCCTGGCTGGCTGCTTGC
TGA

Similarly, by inserting the mutation V287L in the wild-type rat Chrnb2, the base (G) at position c.856 is replaced by the base (C) (c.856G>C) using a forward primer (SEQ ID No. 17) and a reverse primer (SEQ ID No. 18) as indicated below. This results in substitution of the amino acid residue (Val) at position 286 homologous to the β2 subunit of human neuronal nicotinic acetylcholinergic receptor gene for the amino acid residue Leu.

The base sequences of the forward primer (30mer) (SEQ ID No. 17) and the reverse primer (30mer) (SEQ ID No. 18) are indicated as follows:

SEQ ID No. 17:
CTCATCTCCAAGATTATGCCTCCCACCTCC
SEQ ID No. 18:
GGAGGTGGGAGGCATAATCTTGGAGATGAG

The base sequence of the cDNA obtained by the above mutation is indicated as follows:

(SEQ ID No. 19)
GGCCGGGCACTCCAACTCAATGGCGCTGTTCAGCTTCAGCCTTCTTTGGCTGTGCTCAGGGGT
TTTGGGAACTGACACAGAGGAGCGGCTAGTGGAGCATCTCTTAGATCCCTCCCGCTATAACAA
GCTGATTCGTCCAGCTACTAACGGCTCTGAGCTGGTGACTGTACAGCTCATGGTATCATTGGC
TCAGCTCATTAGTGTGCACGAGCGGGAGCAGATCATGACCACCAATGTCTGGCTGACCCAGGA
GTGGGAAGATTACCGCCTCACATGGAAGCCTGAGGACTTCGACAATATGAAGAAAGTCCGGCT
CCCTTCCAAACACATCTGGCTCCCAGATGTGGTTCTATACAACAATGCTGACGGCATGTACGA
AGTCTCCTTCTATTCCAATGCTGTGGTCTCCTATGATGGCAGCATCTTTTGGCTACCACCTGC
CATCTACAAGAGTGCATGCAAGATTGAGGTGAAGCACTTCCCATTTGACCAGCAGAATTGCAC
CATGAAGTTTCGCTCATGGACCTACGACCGTACTGAGATTGACCTGGTGCTCAAAAGTGATGT
GGCCAGTCTGGATGACTTCACACCCAGCGGGGAGTGGGACATCATCGCACTGCCAGGCCGACG
CAACGAGAACCCAGACGACTCCACCTATGTGGACATCACCTATGACTTCATCATTCGTCGCAA
ACCACTCTTCTACACTATCAACCTCATCATCCCCTGCGTACTCATCACCTCGCTGGCCATCCT
GGTCTTCTACCTGCCCTCAGACTGTGGTGAAAAGATGACACTTTGTATTTCTGTGCTGCTAGC
ACTCACGGTGTTCCTGCTGCTCATCTCCAAGATTCTGCCTCCCACCTCCCTCGATGTACCGCT
GGTGGGCAAGTACCTCATGTTTACCATGGTGCTAGTCACCTTCTCCATCGTCACCAGCGTGTG
TGTGCTCAATGTGCACCACCGCTCGCCTACCACGCACACCATGGCCCCCTGGGTCAAGGTGGT
CTTCCTGGAGAAGCTGCCCACCCTGCTCTTCCTGCAGCAGCCACGCCACCGCTGTGCACGTCA
GCGTCTGCGCTTGAGGAGGCGCCAGCGAGAGCGTGAGGGCGCAGGCGCGCTTTTCTTCCGTGA
AGGTCCTGCGGCTGACCCATGTACCTGCTTTGTCAACCCTGCATCAGTGCAGGGCTTGGCTGG
GGCTTTCCGAGCTGAGCCCACTGCAGCCGGCCCGGGGCGCTCTGTGGGGCCATGCAGCTGTGG
CCTCCGGGAAGCAGTGGATGGCGTACGCTTCATTGCGGACCACATGCGAAGTGAGGATGATGA
CCAGAGTGTGAGGGAGGACTGGAAATACGTTGCCATGGTGATCGACCGCCTGTTCCTGTGGAT
CTTTGTCTTTGTCTGTGTCTTTGGGACCGTCGGCATGTTCCTGCAGCCTCTCTTCCAGAACTA
CACTGCCACTACCTTCCTCCACCCTGACCACTCAGCTCCCAGCTCCAAGTGA

In a manner similar to the above mutation, the base residue guanine (G) at position c.856 may be replaced by the base residue adenine (A) (c.856G>A) by inserting the mutation V287M in the wild-type rat Chrnb2, using a forward primer (SEQ ID No. 20) and a reverse primer (SEQ ID No. 21) as indicated below. This insertion results in substitution of the amino acid residue (Val) at position 286 homologous to the β2 subunit of human neuronal nicotinic acetylcholinergic receptor gene for the amino acid residue Met.

The base sequences of the forward primer (30mer) (SEQ ID No. 20) and the reverse primer (30mer) (SEQ ID No. 21) usable for the present invention are indicated as follows:

SEQ ID No. 20:
CTCATCTCCAAGATTCTGCCTCCCACCTCC
SEQ ID No. 22:
GGAGGTGGGAGGCAGAATCTTGGAGATGAG

The base sequence of the cDNA obtained by the above mutation is indicated as follows:

(SEQ ID No. 22)
TGGCCGGGCACTCCAACTCAATGGCGCTGTTCAGCTTCAGCCTTCTTTGGCTGTGCTCAGGGG
TTTTGGGAACTGACACAGAGGAGCGGCTAGTGGAGCATCTCTTAGATCCCTCCCGCTATAACA
AGCTGATTCGTCCAGCTACTAACGGCTCTGAGCTGGTGACTGTACAGCTCATGGTATCATTGG
CTCAGCTCATTAGTGTGCACGAGCGGGAGCAGATCATGACCACCAATGTCTGGCTGACCCAGG
AGTGGGAAGATTACCGCCTCACATGGAAGCCTGAGGACTTCGACAATATGAAGAAAGTCCGGC
TCCCTTCCAAACACATCTGGCTCCCAGATGTGGTTCTATACAACAATGCTGACGGCATGTACG
AAGTCTCCTTCTATTCCAATGCTGTGGTCTCCTATGATGGCAGCATCTTTTGGCTACCACCTG
CCATCTACAAGAGTGCATGCAAGATTGAGGTGAAGCACTTCCCATTTGACCAGCAGAATTGCA
CCATGAAGTTTCGCTCATGGACCTACGACCGTACTGAGATTGACCTGGTGCTCAAAACTGATG
TGGCCAGTCTGGATGACTTCACACCCAGCGGGGAGTGGGACATCATCGCACTGCCAGGCCGAC
GCAACGAGAACCCAGACGACTCCACCTATGTGGACATCACCTATGACTTCATCATTCGTCGCA
AACCACTCTTCTACACTATCAACCTCATCATCCCCTGCGTACTCATCACCTCGCTGGCCATCC
TGGTCTTCTACCTGCCCTCAGACTGTGGTGAAAAGATGACACTTTGTATTTCTGTGCTGCTAG
CACTCACGGTGTTCCTGCTGCTCATCTCCAAGATTATGCCTCCCACCTCCCTCGATGTACCGC
TGGTGGGCAAGTACCTCATGTTTACCATGGTGCTAGTCACCTTCTCCATCGTCACCAGCGTGT
GTGTGCTCAATGTGCACCACCGCTCGCCTACCACGCACACCATGGCCCCCTGGGTCAAGGTGG
TCTTCCTGGAGAAGCTGCCCACCCTGCTCTTCCTGCAGCAGCCACGCCACCGCTGTGCACGTC
AGCGTCTGCGCTTGAGGAGGCGCCAGCGAGAGCGTGAGGGCGCAGGCGCGCTTTTCTTCCGTG
AAGGTCCTGCGGCTGACCCATGTACCTGCTTTGTCAACCCTGCATCAGTGCAGGGCTTGGCTG
GGGCTTTCCGAGCTGAGCCCACTGCAGCCGGCCCGGGGCGCTCTGTGGGGCCATGCAGCTGTG
GCCTCCGGGAAGCAGTGGATGGCGTACGCTTCATTGCGGACCACATGCGAAGTGAGGATGATG
ACCAGAGTGTGAGGGAGGACTGGAAATACGTTGCCATGGTGATCGACCGCCTGTTCCTGTGGA
TCTTTGTCTTTGTCTGTGTCTTTGGGACCGTCGGCATGTTCCTGCAGCCTCTCTTCCAGAACT
ACACTGCCACTACCTTCCTCCACCCTGACCACTCAGCTCCCAGCTCCAAGTGA

The present invention allows a new enzyme site to appear on a mutation site by designing a type of a mutation of interest to be inserted so as to correspond to a base sequence of a codon located at the position of the mutation site in the manner as described above.

For instance, in the case of Chrna4, the mutation of the mutated 879-880insTTA causes an appearance of the cleavage site (T↓TAA) by restriction enzyme Tru9 I (MseI). In the case of Chrnb2, the mutation of the mutated V287L or V287M causes an appearance of the cleavage site (G↓ANTG) by restriction enzyme Hint If. In the above description, the arrow symbol (↓) denotes a position of cleavage by a restriction enzyme. It is to be noted herein that the kinds of the restriction enzyme and restriction sites are not restricted to the above ones and may be selected appropriately by changing mutations.

In accordance with the present invention, a nucleotide consisting of a particular base sequence can be introduced as a probe into the above cDNA. The probe may be preferably prepared so as to have a base sequence thoroughly or substantially thoroughly different from the original base sequence yet encode the amino acid sequence identical thereto. The base number of the probe may be in the length ranging generally from approximately 30 bp to 200 bp, preferably from approximately 50 bp to 150 bp, more preferably from approximately 60 bp to 120 bp. These nucleotides may be prepared by conventional methods including DNA synthesis, as used commonly in the art.

More specifically, for instance, for the mutated cDNA of the Chrna4 gene, a nucleotide having the following base sequence (118 bp) can be prepared so as to have a base sequence different from the base sequence starting with position c.104 and ending at position c.221 of the cDNA, yet coding for the same amino acid sequence.

(SEQ ID No. 23)

GGGCTCACGCCGAAGAACGCCTGCTCAAAAGGCTGTTTTCTGGCTATAAT
AAATGGTCCCGCCCCGTGGCTAACATTTCCGACGTCGTGCTGGTGCGGTT
CGGATTATCTATCGCTCA

Similarly, for the mutated cDNA of the Chrnb2 gene, a nucleotide having the following base sequence (62 bp) can be prepared as a probe so as to have a base sequence different from the base sequence starting with position c.1171 and ending at position c.1232 of the cDNA, yet coding for the same amino acid sequence.

(SEQ ID No. 24)

AACCCCGCCTCCGTCCAAGGACTCGCCGGCGCCTTTAGGGCCGAACCTAC
CGCCGCTGGCCC

Thereafter, the resulting nucleotide probe may be hybridized and inserted into a site of a restriction enzyme in a conventional manner. For instance, the probe indicated below as SEQ ID No. 22 may be inserted into a designated site on the mutated cDNA of the Chrna4 subunit gene having the above base sequence, e.g., a site of Sma I and Bpul102 (alternatively called as Esp I or Cel II site) of an appropriate vector such as pCRII-TOPO vector, etc., using a probe insertion method as conventionally used in the art. Alternatively, for instance, the probe indicated below as SEQ ID No. 22 may also be inserted into a designated site of the mutated cDNA of the Chrnb2 subunit gene having the above base sequence, e.g., a site of Hinc I and Sma I of an appropriate vector such as pCRII-TOPO vector, etc., using a probe insertion method as conventionally used in the art. At each step, the base sequence is confirmed by sequencing.

The base sequence (SEQ ID No. 25) of the mutated gene cDNA (S282FPB) with the probe (SEQ ID No. 23) inserted into the mutated cDNA (S282F) (i.e., S280F in humans) of the rat Chrna4 subunit gene is indicated as below. It is to be noted herein that the mutation of c.845C>T and c.846G>C causes the bases TCG at positions 844-846 to be mutated into the mutated bases TTC as enclosed by circle, and the probe portion (118 bp) is indicated as underlined at positions 104-221.

(SEQ ID No. 25)
ATGGCCAATTCGGGCACCGGGGCGCCGCCGCCGCTGCTGCTACTGCCGCTGCTGCTGCTCCTA
GGGACCGGCCTCTTGCCTGCTAGCAGCCACATAGAGACCCGGGCTCACGCCGAAGAACGCCTG
CTCAAAAGGCTGTTTTCTGGCTATAATAAATGGTCCCGCCCCGTGGCTAACATTTCCGACGTC
GTGCTGGTGCGGTTCGGATTATCTATCGCTCAGCTCATTGACGTGGACGAGAAGAACCAGATG
ATGACAACCAACGTGTGGGTGAAGCAGGAGTGGCACGACTACAAGCTGCGCTGGGACCCTGGT
GACTACGAGAATGTCACCTCCATCCGCATCCCCTCTGAACTCATCTGGAGGCCTGACATCGTC
CTCTACAACAATGCGGATGGAGACTTTGCAGTCACCCACCTGACCAAGGCCCACCTGTTCTAT
GACGGAAGGGTGCAGTGGACACCCCCAGCCATCTATAAGAGCTCCTGCAGCATCGACGTCACC
TTCTTCCCCTTTGACCAGCAGAACTGTACCATGAAGTTTGGATCCTGGACCTACGACAAGGCC
AAGATTGACTTAGTGAGCATGCATAGCCGTGTGGACCAACTGGACTTCTGGGAAAGTGGGGAG
TGGGTCATCGTGGATGCTGTGGGCACCTACAACACCAGGAAGTACGAGTGCTGTGCCGAGATC
TATCCTGACATCACCTATGCCTTCATCATCCGACGGCTGCCGCTATTCTACACCATCAACCTC
ATCATCCCGTGCCTGCTCATCTCCTGTCTCACCGTGCTGGTCTTCTATCTGCCTTCAGAGTGT
GGCGAGAAGGTCACACTGTGCATCTTCGTGCTGCTTTCTCTCACCGTCTTCCTGCTGCTCATC
ACCGAGATCATCCCGTCCACCTCGCTGGTCATCCCGCTCATCGGCGAGTACCTCCTCTTCACC
ATGATCTTCGTCACCCTCTCCATCGTCATCACCGTCTTCGTGCTCAATGTGCACCACCGCTCG
CCACGCACACACACGATGCCCGCCTGGGTGCGTAGAGTCTTCCTGGACATCGTGCCTCGCCTC
CTCTTCATGAAGCGCCCCTCTGTGGTCAAAGACAACTGCCGGAGACTTATTGAGTCCATGCAC
AAGATGGCCAACGCCCCCCGCTTCTGGCCAGAGCCTGTGGGCGAGCCCGGCATCTTGAGTGAC
ATCTGCAACCAAGGTCTGTCACCTGCCCCAACTTTCTGCAACCCCACGGACACAGCAGTCGAG
ACCCAGCCTACGTGCAGGTCACCCCCCCTTGAGGTCCCTGACTTGAAGACATCAGAGGTTGAG
AAGGCCAGTCCCTGTCCATCGCCTGGCTCCTGTCCTCCACCCAAGAGCAGCAGTGGGGCTCCA
ATGCTCATCAAAGCCAGGTCCCTGAGTGTCCAGCATGTGCCCAGCTCCCAAGAAGCAGCAGAA
GATGGCATCCGCTGCCGGTCTCGGAGTATCCAGTACTGTGTTTCCCAAGATGGAGCTGCCTCC
CTGGCTGACAGCAAGCCCACCAGCTCCCCGACCTCCCTGAAGGCCCGTCCATCCCAGCTTCCC
GTGTCAGACCAGGCCTCTCCATGCAAATGCACATGCAAGGAACCATCTCCTGTGTCCCCAGTC
ACTGTGCTCAAGGCGGGAGGCACCAAAGCACCTCCCCAACACCTGCCCCTGTCACCAGCCCTG
ACACGGGCAGTAGAAGGCGTCCAGTACATTGCAGACCACCTCAAGGCAGAAGACACTGACTTC
TCGGTGAAGGAGGACTGGAAATACGTGGCCATGGTCATTGACCGAATCTTCCTCTGGATGTTC
ATCATTGTCTGCCTTCTGGGCACTGTGGGACTCTTCCTGCCTCCCTGGCTGGCTGCTTGCTGA

The base sequence (SEQ ID No. 26) of the mutated gene cDNA (S286LPB) with the probe (SEQ ID No. 23) inserted into the mutated cDNA (S286L) (i.e., S284L in humans) of the rat Chrna4 subunit gene is indicated as below. It is to be noted herein that the mutation of c.856T>C and c.857C>T causes the bases TCT at positions 856-858 to be mutated into the mutated CTT as enclosed by circle, and the probe portion (118 bp) is indicated as underlined at positions 104-221.

(SEQ ID No. 26)
ATGGCCAATTCGGGCACCGGGGCGCCGCCGCCGCTGCTGCTACTGCCGCTGCTGCTGCTCCTA
GGGACCGGCCTCTTGCCTGCTAGCAGCCACATAGAGACCCGGGCTCACGCCGAAGAACGCCTG
CTCAAAAGGCTGTTTTCTGGCTATAATAAATGGTCCCGCCCCGTGGCTAACATTTCCGACGTC
GTGCTGGTGCGGTTCGGATTATCTATCGCTCAGCTCATTGACGTGGACGAGAAGAACCAGATG
ATGACAACCAACGTGTGGGTGAAGCAGGAGTGGCACGACTACAAGCTGCGCTGGGACCCTGGT
GACTACGAGAATGTCACCTCCATCCGCATCCCCTCTGAACTCATCTGGAGGCCTGACATCGTC
CTCTACAACAATGCGGATGGAGACTTTGCAGTCACCCACCTGACCAAGGCCCACCTGTTCTAT
GACGGAAGGGTGCAGTGGACACCCCCAGCCATCTATAAGAGCTCCTGCAGCATCGACGTCACC
TTCTTCCCCTTTGACCAGCAGAACTGTACCATGAAGTTTGGATCCTGGACCTACGACAAGGCC
AAGATTGACTTAGTGAGCATGCATAGCCGTGTGGACCAACTGGACTTCTGGGAAAGTGGGGAG
TGGGTCATCGTGGATGCTGTGGGCACCTACAACACCAGGAAGTACGAGTGCTGTGCCGAGATC
TATCCTGACATCACCTATGCCTTCATCATCCGACGGCTGCCGCTATTCTACACCATCAACCTC
ATCATCCCGTGCCTGCTCATCTCCTGTCTCACCGTGCTGGTCTTCTATCTGCCTTCAGAGTGT
GGCGAGAAGGTCACACTGTGCATCTCGGTGCTGCTTCTTCTCACCGTCTTCCTGCTGCTCATC
ACCGAGATCATCCCGTCCACCTCGCTGGTCATCCCGCTCATCGGCGAGTACCTCCTCTTCACC
ATGATCTTCGTCACCCTCTCCATCGTCATCACGGTCTTCGTGCTCAATGTGCACCACCGCTCG
CCACGCACACACACGATGCCCGCCTGGGTGCGTAGAGTCTTCCTGGACATCGTGCCTCGCCTC
CTCTTCATGAAGCGCCCCTCTGTGGTCAAAGACAACTGCCGGAGACTTATTGAGTCCATGCAC
AAGATGGCCAACGCCCCCCGCTTCTGGCCAGAGCCTGTGGGCGAGCCCGGCATCTTGAGTGAC
ATCTGCAACCAAGGTCTGTCACCTGCCCCAACTTTCTGCAACCCCACGGACACAGCAGTCGAG
ACCCAGCCTACGTGCAGGTCACCCCCCCTTGAGGTCCCTGACTTGAAGACATCAGAGGTTGAG
AAGGCCAGTCCCTGTCCATCGCCTGGCTCCTGTCCTCCACCCAAGAGCAGCAGTGGGGCTCCA
ATGCTCATCAAAGCCAGGTCCCTGAGTGTCCAGCATGTGCCCAGCTCCCAAGAAGCAGCAGAA
GATGGCATCCGCTGCCGGTCTCGGAGTATCCAGTACTGTGTTTCCCAAGATGGAGCTGCCTCC
CTGGCTGACAGCAAGCCCACCAGCTCCCCGACCTCCCTGAAGGCCCGTCCATCCCAGCTTCCC
GTGTCAGACCAGGCCTCTCCATGCAAATGCACATGCAAGGAACCATCTCCTGTGTCCCCAGTC
ACTGTGCTCAAGGCGGGAGGCACCAAAGCACCTCCCCAACACCTGCCCCTGTCACCAGCCCTG
ACACGGGCAGTAGAAGGCGTCCAGTACATTGCAGACCACCTCAAGGCAGAAGACACTGACTTC
TCGGTGAAGGAGGACTGGAAATACGTGCCCATGGTCATTGACCGAATCTTCCTCTGGATGTTC
ATCATTGTCTGCCTTCTGGGCACTGTGGGACTCTTCCTGCCTCCCTGGCTGGCTGCTTGCTGA

The base sequence (SEQ in No. 27) of the mutated gene cDNA (S282FPB) with the probe (SEQ ID No. 23) inserted into the mutated cDNA (S79-880insL) (i.e., S284L in humans) of the rat Chrna4 subunit gene is indicated as below. In the base sequence below, the mutation of c.845C>T and c.846G>C causes the bases TCG at positions 844-846 to the mutated TTC is indicated as enclosed by circle, and the probe portion (118 bp) at positions 104-221 is indicated as underlined. The mutation regarding human beings is represented by S280F.

(SEQ ID No. 27)
ATGGCCAATTCGGGCACCGGGGCGCCGCCGCCGCTGCTGCTACTGCCGCTGCTGCTGCTCCTA
GGGACCGGCCTCTTGCCTGCTAGCAGCCACATAGAGACCCGGGCTCACGCCGAAGAACGCCTG
CTCAAAAGGCTGTTTTCTGGCTATAATAAATGGTCCCGCCCCGTGGCTAACATTTCCGACGTC
GTGCTGGTGCGGTTCGGATTATCTATCGCTCAGCTCATTGACGTGGACGAGAAGAACCAGATG
ATGACAACCAACGTGTGGGTGAAGCAGGAGTGGCACGACTACAAGCTGCGCTGGGACCCTGGT
GACTACGAGAATGTCACCTCCATCCGCATCCCCTCTGAACTCATCTGGAGGCCTGACATCGTC
CTCTACAACAATGCGGATGGAGACTTTGCAGTCACCCACCTGACCAAGGCCCACCTGTTCTAT
GACGGAAGGGTGCAGTGGACACCCCCAGCCATCTATAAGAGCTCCTGCAGCATCGACGTCACC
TTCTTCCCCTTTGACCAGCAGAACTGTACCATGAAGTTTGGATCCTGGACCTACGACAAGGCC
AAGATTGACTTAGTGAGCATGCATAGCCGTGTGGACCAACTGGACTTCTGGGAAAGTGGGGAG
TGGGTCATCGTGGATGCTGTGGGCACCTACAACACCAGGAAGTACGAGTGCTGTGCCGAGATC
TATCCTGACATCACCTATGCCTTCATCATCCGACGGCTGCCGCTATTCTACACCATCAACCTC
ATCATCCCGTGCCTGCTCATCTCCTGTCTCACCGTGCTGGTCTTCTATCTGCCTTCAGAGTGT
GGCGAGAAGGTCACACTGTGCATCTCGGTGCTGCTTTCTCTCACCGTCTTCCTGCTGCTGCTA
ATCACCGAGATCATCCCGTCCACCTCGCTGGTCATCCCGCTCATCGGCGAGTACCTCCTCTTC
ACCATGATCTTCGTCACCCTCTCCATCGTCATCACGGTCTTCGTGCTCAATGTGCACCACCGC
TCGCCACGCACACACACGATGCCCGCCTGGGTGCGTAGAGTCTTCCTGGACATCGTGCCTCGC
CTCCTCTTCATGAAGCGCCCCTCTGTGGTCAAAGACAACTGCCGGAGACTTATTGAGTCCATG
CACAAGATGGCCAACGCCCCCCGCTTCTGGCCAGAGCCTGTGGGCGAGCCCGGCATCTTGAGT
GACATCTGCAACCAAGGTCTGTCACCTGCCCCAACTTTCTGCAACCCCACGGACACAGCAGTC
GAGACCCAGCCTACGTGCAGGTCACCCCCCCTTGAGGTCCCTGACTTGAAGACATCAGAGGTT
GAGAAGGCCAGTCCCTGTCCATCGCCTGGCTCCTGTCCTCCACCCAAGAGCAGCAGTGGGGCT
CCAATGCTCATCAAAGCCAGGTCCCTGAGTGTCCAGCATGTGCCCAGCTCCCAAGAAGCAGCA
GAAGATGGCATCCGCTGCCGGTCTCGGAGTATCCAGTACTGTGTTTCCCAAGATGGAGCTGCC
TCCCTGGCTGACAGCAAGCCCACCAGCTCCCCGACCTCCCTGAAGGCCCGTCCATCCCAGCTT
CCCGTGTCAGACCAGGCCTCTCCATGCAAATGCACATGCAAGGAACCATCTCCTGTGTCCCCA
GTCACTGTGCTCAAGGCGGGAGGCACCAAAGCACCTCCCCAACACCTGCCCCTGTCACCAGCC
CTGACACGGGCAGTAGAAGGCGTCCAGTACATTGCAGACCACCTCAAGGCAGAAGACACTGAC
TTCTCGGTGAAGGAGGACTGGAAATACGTGGCCATGGTCATTGACCGAATCTTCCTCTGGATG
TTCATCATTGTCTGCCTTCTGGGCACTGTGGGACTCTTCCTGCCTCCCTGGCTGGCTGCTTGC
TGA

The base sequence (SEQ ID No. 28) of the mutated gene cDNA (V286L) with a probe (SEQ ID No. 24) inserted into the mutated cDNA of the rat Chrnb2 subunit gene is indicated as below.

(SEQ ID No. 28)
GGCCGGGCACTCCAACTCAATGGCGCTGTTCAGCTTCAGCCTTCTTTGGCTGTGCTCAGGGGT
TTTGGGAACTGACACAGAGGAGCGGCTAGTGGAGCATCTCTTAGATCCCTCCCGCTATAACAA
GCTGATTCGTGCAGCTACTAACGGCTCTGAGCTGGTGACTGTACAGCTCATGGTATCATTGGC
TCAGCTCATTAGTGTGCACGAGCGGGAGCAGATCATGACCACCAATGTCTGGCTGACCCAGGA
GTGGGAAGATTACCGCCTCACATGGAAGCCTGAGGACTTCGACAATATGAAGAAAGTCCGGCT
CCCTTCCAAACACATCTGGCTCCCAGATGTGGTTCTATACAACAATGCTGACGGCATGTACGA
AGTCTCCTTCTATTCCAATGCTGTGGTCTCCTATGATGGCAGCATCTTTTGGCTACCACCTGC
CATCTACAAGAGTGCATGCAAGATTGAGGTGAAGCACTTGCCATTTGACCAGCAGAATTGCAC
CATGAAGTTTCGCTCATGGACCTACGACCGTACTGAGATTGACCTGGTGCTCAAAAGTGATGT
GGCCAGTCTGGATGACTTCACACCCAGCGGGGAGTGGGACATCATCGCACTGCCAGGCCGACG
CAACGAGAACCCAGACGACTCCACCTATGTGGACATCACCTATGACTTCATCATTCGTCGCAA
ACCACTCTTCTACACTATCAACCTCATCATCCCCTGCGTACTCATCACCTCGCTGGCCATCCT
GGTCTTCTACCTGCCCTCAGACTGTGGTGAAAAGATGACACTTTGTATTTCTGTGCTGCTAGC
ACTCACGGTGTTCCTGCTGCTCATCTCCAAGATTCTGCCTCCCACCTCCCTCGATGTACCGCT
GGTGGGCAAGTACCTCATGTTTACCATGGTGCTAGTCACCTTCTCCATCGTCACCAGCGTGTG
TGTGCTCAATGTGCACCACCGCTCGCCTACCACGCACACCATGGCCCCCTGGGTCAAGGTGGT
CTTCCTGGAGAAGCTGCCCACCCTGCTCTTCCTGCAGCAGCCACGCCACCGCTGTGCACGTCA
GCGTCTGCGCTTGAGGAGGCGCCAGCGAGAGCGTGAGGGCGCAGGCGCGCTTTTCTTCCGTGA
AGGTCCTGCGGCTGACCCATGTACCTGCTTTGTCAACCCCGCCTCCGTCCAAGGACTCGCCGG
CGCCTTTAGGGCCGAACCTACCGCCGCTGGCCCGGGGCGCTCTGTGGGGCCATGCAGCTGTGG
CCTCCGGGAAGCAGTGGATGGCGTACGCTTCATTGCGGACCACATGCGAAGTGAGGATGATGA
CCAGAGTGTGAGGGAGGACTGGAAATACGTTGCCATGGTGATCGACCGCCTGTTCCTGTGGAT
CTTTGTCTTTGTCTGTGTCTTTGGGACCGTCGGCATGTTCCTGCAGCCTCTCTTCCAGAACTA
CACTGCCACTACCTTCCTCCACCCTGACCACTCAGCTCCCAGCTCCAAGTGA

The base sequence (SEQ ID No. 29) of the mutated gene cDNA (V286M) with a probe (SEQ ID No. 24) inserted into the mutated cDNA of the rat Chrnb2 subunit gene is indicated as below.

(SEQ ID No. 29)
TGGCCGGGCACTCCAACTCAATGGCGCTGTTCAGCTTCAGCCTTCTTTGGCTGTGCTCAGGGG
TTTTGGGAACTGACACAGAGGAGCGGCTAGTGGAGCATCTCTTAGATCCCTCCCGCTATAACA
AGCTGATTCGTCCAGCTACTAACGGCTCTGAGCTGGTGACTGTACAGCTCATGGTATCATTGG
CTCAGCTCATTAGTGTGCACGAGCGGGAGCAGATCATGACCACCAATGTCTGGCTGACCCAGG
AGTGGGAAGATTACCGCCTCACATGGAAGCCTGAGGACTTCGACAATATGAAGAAAGTCCGGC
TCCCTTCCAAACACATCTGGCTCCCAGATGTGGTTCTATACAACAATGCTGACGGCATGTACG
AAGTCTCCTTCTATTCCAATGCTGTGGTCTCCTATGATGGCAGCATCTTTTGGCTACCACCTG
CCATCTACAAGAGTGCATGCAAGATTGAGGTGAAGCACTTCCCATTTGACCAGCAGAATTGCA
CCATGAAGTTTCGCTCATGGACCTACGACCGTACTGAGATTGACCTGGTGCTCAAAAGTGATG
TGGCCAGTCTGGATGACTTCACACCCAGCGGGGAGTGGGACATCATCGCACTGCCAGGCCGAC
GCAACGAGAACCCAGACGACTCCACCTATGTGGACATCACCTATGACTTCATCATTCGTCGCA
AACCACTCTTCTACACTATCAACCTCATCATCCCCTGCGTACTCATCACCTCGCTGGCCATCC
TGGTCTTCTACCTGCCCTCAGACTGTGGTGAAAAGATGACACTTTGTATTTCTGTGCTGCTAG
CACTCACGGTGTTCCTGCTGCTCATCTCCAAGATTATGCCTCCCACCTCCCTCGATGTACCGC
TGGTGGGCAAGTACCTCATGTTTACCATGGTGCTAGTCACCTTCTCCATCGTCACCAGCGTGT
GTGTGCTCAATGTGCACCACCGCTCGCCTACCACGCACACCATGGCCCCCTGGGTCAAGGTGG
TCTTCCTGGAGAAGCTGCCCACCCTGCTCTTCCTGCAGCAGCCACGCCACCGCTGTGCACGTC
AGCGTCTGCGCTTGAGGAGGCGCCAGCGAGAGCGTGAGGGCGCAGGCGCGCTTTTCTTCCGTG
AAGGTCCTGCGGCTGACCCATGTACCTGCTTTGTCAACCCCGCCTCCGTCCAAGGACTCGCCG
GCGCCTTTAGGGCCGAACCTACCGCCGCTGGCCCGGGGCGCTCTGTGGGGCCATGCAGCTGTG
GCCTCCGGGAAGCAGTGGATGGCGTACGCTTCATTGCGGACCACATGCGAAGTGAGGATGATG
ACCAGAGTGTGAGGGAGGACTGGAAATACGTTGCCATGGTGATCGACCGCCTGTTCCTGTGGA
TCTTTGTCTTTGTCTGTGTCTTTGGGACCGTCGGCATGTTCCTGCAGCCTCTCTTCCAGAACT
ACACTGCCACTACCTTCCTCCACCCTGACCACTCAGCTCCCAGCTCCAAGTGA

By using the rat Chrna4 or Chrnb2 cDNA with the mutation and the probe produced by the above techniques, the model non-human mammalian animals for human epilepsy according to the present invention may be produced by non-human mammalian animals-producing methods known per se in the art. Such methods may include, for example, a transgenic animals-producing method involved in introduction of a gene of interest into an animal individual, a method of genetic replacement animals involved in replacement of a target gene by a gene of interest, a clone producing method involved in using a nucleus of a cell produced by modifying a gene of interest, and so on.

In this description, there is described the transgenic animals-producing method for producing transgenic animals such as rats, for example, by introducing the cDNA of the mutated gene as an objective gene into a fertilized egg of the individual animal.

First, an expression vector is formed, which includes a DNA fragment encoding a mutated gene having the above mutation in the gene homologous to the α4 subunit (CHRNA4) or the β2 subunit (CHRNB2) of the neuronal nicotinic acetylcholinergic receptor gene associated with human autosomal dominant nocturnal frontal lobe epilepsy.

More specifically, the expression vector can be constructed by isolating the DNA fragment containing the α4 subunit (Chrna4) or the β2 subunit (Chrnb2) of the neuronal nicotinic acetylcholinergic receptor gene associated with human autosomal dominant nocturnal frontal lobe epilepsy from the non-human mammalian animal and inserting an exogenous gene, etc. into the DNA fragment. Such an exogenous gene may include, for example, preferably a marker gene, particularly preferably an antibiotic-resistant gene such as a neomycin-resistant gene and so on. In the event where such an antibiotic-resistant gene is inserted, a homologously recombinant cell line can be selected simply by incubating the cell line in culture medium containing the corresponding antibiotic. Further, in order to achieve a more efficient selection, it is preferred to connect a thymidine kinase gene or the like to the expression vector. This can exclude the cell lines with no homologous recombination. Moreover, it is possible to exclude the non-homologously recombinant cell lines by connecting diphteria toxin A fragment (DT-A) gene or the like to the expression vector. Such vectors may include, for example, pCI-neo, pMCIneo, pXT1, pSG5, pcDNA3.neo, pLITMUS28, pcDNAIamp-pcDNA3, and so on.

The above DNA fragment may be preferably inserted in the non-human mammalian animal by using the expression vector as an insertion gene to which the DNA fragment is connected at a location downstream of an appropriate promoter. As a promoter, there may be used any promoter that can initiate transcription in the cells of the non-human mammalian animal to which a coding DNA of the receptor subunit gene is introduced. The promoter is not limited to a particular one and may include, for example, a gene derived from a virus such as simian virus 40, polyomavirus, adenovirus 2, human cytomegalovirus, retrovirus, or the like.

The expression vector to be used for the present invention may preferably possess a poly-A additional signal necessary for polyadenylation at the 3′-terminus of mRNA at a position located downstream of the DNA encoding the mutated gene of the α4 subunit (Chrna2) or the β2 subunit (Chrnb2) of the neuronal nicotinic acetylcholinergic receptor gene of a non-human mammalian animal. As such a poly-A additional signal, there may be used, for example, a poly-A additional signal of the late gene or the early gene of SV40 contained in each gene derived from the above virus, etc. Moreover, in order to more highly express the gene of interest, the expression vector may preferably contain a splicing signal, an enhancer region, or a portion of intron of each gene, ligated at a 5′-downstream location of the promoter region or between the promoter region and a translation region or at a 3′-downstream location of the translation region.

In en embodiment according to the present invention, for instance, the mutated rat Chrna4 or Chrnb2 cDNA with the above mutation and the probe introduced thereinto is transferred to the expression vector using restriction enzymes Xho I and Not/and then cleaved with restriction enzymes SnaBI and NaeI. For instance, on one hand, in the event where the mutated rat Chrna4 cDNA is to be transferred to an expression vector having a PDGF promoter derived from a pCI-neo vector, it can be transferred with restriction enzymes XhoI and Not I and then cleaved with restriction enzymes SnaBI and Nae I. Further, in the event where the mutated rat Chrnb2 cDNA is to be transferred thereto, it can be transferred with restriction enzymes Xho I and Sal I and then cleaved with restriction enzymes SnaBI and DraIII. This allows a fragment consisting of the PDGF promoter, the mutated rat Chrna4 or Chrnb2 cDNA and the poly-A portion can be isolated and purified as a DNA construct by gel processing.

In an embodiment according to the present invention, for instance, the DNA construct isolated and purified as described above is then introduced into differentially totipotent cells of the non-human mammalian animal leading to the production of the non-human mammalian animal as a model animal of interest. Examples of the totipotent cells may include, for example, fertilized eggs, early embryos, embryonic stem cells, and so on. The fertilized eggs to be used for introduction of the DNA construct may be obtained, for example, by intraperitoneally administering the animal with an ovulatory drug such as gonadotropic hormone (PMS), etc., inducing superovulation in female animals and collecting fertilized eggs usable for introduction of the DNA construct. A pseudopregnant female animal with the fertilized eggs is crossed with a male mouse castrated by vasoligation, etc. and the oviduct of the pseudopregnant female animal was removed to collect the eggs by retrieving them under a microscope.

To the resulting fertilized eggs is then introduced the DNA construct. Although various methods are known for introducing the DNA construct to the fertilized eggs, microinjection is preferred in order to promote a production efficiency of transgenic animals and a transmission efficiency of the inserted gene to the subsequent generations. The fertilized egg with the mutated gene injected thereinto is transplanted to the oviduct of a foster mother resulting in a delivery of offspring. After delivery, the DNA is then extracted from the somatic cells of a portion of the body (e.g., the tail tip) to confirm the presence of the transferred gene by southern blotting or PCR in order to select the transgenic animals of interest from the delivered offspring individuals, which possess the introduced gene in the genome of the somatic cells.

The individual (i.e., a heterozygote) with the presence of the inserted gene confirmed as above is determined as a founder (F0). As the introduced gene is transmitted by 50% to its offspring (F1). By crossing the F1 female individual with the F1 male individual, individuals (F2) having the gene in both of the diploid chromosomes can be produced.

In contrast, the transgenic method for forming the model non-human mammalian animals for human epilepsy according to the present invention can introduce the mutated gene encoding a mutated Chrna4 or Chrnb2 gene at an optional position of the chromosomal DNA in such a state that the endogenous gene (Chrna4 or Chrnb2 gene) is present in a normal state. Therefore, the resulting human epilepsy model animals according to the present invention can simultaneously produce normal Chrna4 or Chrnb2 gene as well as mutated Chrna4 or Chrnb2 gene, respectively. As the neuronal nicotinic acetycholinergic receptor is a protein that functions as an ion channel (consisting of two α subunits and three β subunits), the channel function may be altered if either of the subunits would be mutated. Therefore, as in the present invention, it is considered to be appropriate that the human epilepsy model animals demonstrate epileptic seizures of a human type upon simultaneous expression of both the normal subunit and the mutated subunit.

The model non-human mammalian animals for human epilepsy according to the present invention possess supeior properties of spontaneously developing epileptic seizures during sleep as in the case of human autosomal dominant nocturnal frontal lobe epilepsy as will be described under Examples below.

The individuals with the gene of interest incorporated in the chromosomes of all the cells may be selected usually by preparing DNA from a portion of tissues such as blood tisues, epithelial tissues and so on and subjecting the resulting DNA to DNA sequencing to confirm the presence of the introduced gene at a DNA level. The selection of the recombinant indivisual by the DNA sequencing as above, however, requires laborious work and a long labor time as well as a large amount of expenses.

In other words, in the event where disease model animals with genetic abnormality are produced, a homologous recombinant is required to be determined in an accurate way. At this end, the conventional method for producing disease model animals requires sequencing of a portion of the gene of an indivisual and expressing mRNA of the introduced gene. It is not easy, however, to express mRNA of the introduced gene by RT-PCR, in situ hybridization, and so on.

Therefore, the present invention is devised so as to easily distinguishing the recombinants by introducing the mutated gene as described above and replacing a portion of the mutated gene by a probe. Moreover, the present invention enables an easy distinction of the recombinants by causing a new restriction site of a restriction enzyme to appear by the mutation as above. It is to be understood herein that this approach is not restricted to specific embodiments as will be described below and it can be commonly applied to every mutation regardless of types of mutations and kinds of restriction enzymes, etc. In addition, the present invention does not particularly restrict the type of the mutation for introduction into a specific site of the cDNA clone to a specific one.

More specifically, in order to allow for easy selection of such recombinant individuals, the present invention is provided in such a manner that a new restriction site of the restriction enzyme is caused to appear by introducing the mutation such as, for example, S280L, S284L, S291-292L, V287L, V287M, etc., into the DNA fragment encoding the α4 subunit (Chrna4) or the β2 subunit (Chrnb2) of the neuronal nicotinic acetylcholinergic receptor gene of the non-human mammalian animal. The appearance of the new restriction site of the restriction enzyme can easily distinguish the recombinants upon formation of the recombinants.

In an embodiment of the present invention, the base sequence of a portion of the mutated cDNA is replaced by a probe with a base sequence that is substantially thoroughly different therefrom yet encodes an amino acid sequence identical thereto, and the probe is introduced at the site identical to the site of the original base sequence. Therefore, the recombinant individuals can be easily distinguished using the restriction enzymes for the introduced probe. Moreover, the mRNA expressed from the recombinant gene from the original rat Chrna4 mRNA or Chrnb2 mRNA can be detected easily by techniques, such as RT-PCR or in situ hybridization, etc.

Further, for instance, a restriction site of restriction enzyme Hinf If is caused to occur by introduction of the mutation (c.856G>C) or (c.856G>A) to the homologous gene Chrnb2 of the β2 subunit (CHRNB2) of the neuronal nicotinic acetylcholinergic receptor gene. Therefore, the region containing this restriction site can be identified using the restriction enzyme Hinf If by PCR-RFLP (restriction fragment length polymorphism), etc. This indicates that, on one hand, an individual with the given homologous recombination of interes possesses the restriction site of the restriction enzyme Hinf If so that the restriction site involved is cleaved by the restriction enzyme Hinf If and that, on the other hand, an individual with no given homologous recombination does not possess the restriction site of the restriction enzyme Hinf If so that no cleavage is caused to occur by the restriction enzyme Hinf If. Therefore, the present invention allows a ready distinction of the recombinants by the mutation of the homologous gene Chrna4 or Chrnb2 of the α4 subunit CHRNA4 or the β4 subunit CHRNB2, respectively, of the neuronal nicotinic acetylcholinergic receptor gene so as to cause an appearance of a restriction site of the corresponding restriction enzyme.

It is noted herein that the PCR-RFLP method to be used herein is a method well known per se in the art and this method enables a detection of a presence or absence of a restriction site of a restriction enzyme by amplifying the involved portions by PCR, digestion of the amplified product with a given restriction enzyme such as True9I or Hinf If, and determination of the digested DNA fragment size using electrophoresis, etc.

The present invention will be described more in detail by way of examples. It is to be understood that the present invention is not interpreted as being restricted in any respect by the following examples and that the following examples are described solely for illustrative purposes to explain the invention more specifically.

### Example 1

First, PCR was performed using a primer set consisting of two primers designed based on a known cDNA sequence of rat Chrna4 using Rat Brain QUICK-Clone cDNA (CLONTECH; Mountain View, Calif.) as a template, namely,

(40 mer; SEQ ID NO: 3)

a forward primer (BN-Rat CHRNA4 cDNA-F):
AGATCTCGCGAAGCTTCACCATGGCCAATTCGGGCACCGG,
and

(38 mer; SEQ ID NO: 4)

a reverse primer (Rat CHRNA4 cDNA-BX-R):
AGATCTAGATCAGCAAGCAGCCAGCCAGGGAGGCAGGA.

The PCR product was purified and subcloned into a pCRII-TOPO vector (Invitorogen; Carlsbad, Calif.). The resulting clone was sequenced to confirm the base sequence of the Chrna4 cDNA. The rat base sequence information is available under accession number L31620 (NCBI).

Mutation was introduced into the clone using QuikChange Site-Directed Mutagenesis Kit (STRATAGENE: La Jolla, Calif.). First, the mutation was performed by mutating cytosine C at position 845 of the cDNA base sequence to thymine T and guanine G at position 846 thereof to cytosine C, respectively, using:

(29 mer; SEQ ID NO: 7)

r845T846C-sen: CACACTGTGCATCTTCGTGCTGCTTTCTC;
and

(29 mer; SEQ ID NO: 8)

r845T846C-ant: GAGAAAGCAGCACGAAGATGCACAGTGTG.

The base sequence of the mutated cDNA is as shown in SEQ ID NO: 9. This allows a mutation of amino acid residue Ser to amino acid residue Phe mutation at position p.282 homologous to the α4 subunit of the human neuronal nicotinic acetylcholinergic receptor gene.

A probe was prepared, which consists of a 118 bp nucleotide (SEQ ID NO:23) having a base sequence thoroughly different from its original base sequence yet coding for the same amino acid sequence at positions c.104-c.221 of the mutated cDNA.

(SEQ ID NO: 23)

GGGCTCACGCCGAAGAACGCCTGCTCAAAAGGCTGTTTTCTGGCTATAAT
AAATGGTCCCGCCCCGTGGCTAACATTTCCGACGTCGTGCTGGTGCGGTT
CGGATTATCTATCGCTCA

After hybridization of the mutated cDNA with the introduced probe, the hybridized cDNA was inserted into a pCRII-TOPO vector at the given restriction site of the Chrna4 cDNA inserted in the vector using the restriction enzyme sites SmaI and Bpul102I. The insert was sequenced in each step, and the base sequence was confirmed.

The rat Chrna4 cDNA with the mutation (S286L) was transferred to an expression vector with a PDGF promoter derived from a pCI-neo vector using the restriction enzyme sites XhoI and NotI, and then cut with restriction enzymes SnaBI and NaeI. A fragment including the PDGF promoter, the rat Chrna4 cDNA and a poly A portion was isolated by gel processing and purified. The isolated and purified DNA was microinjected into fertilized eggs, and over 200 eggs in good condition were transplanted into the oviduct of recipient females. The animals were allowed to give natural birth to recombinants.

### Example 2

In substantially the same manner as in Example 1, the mutation was performed by mutating thymine T to cytosine C at position 856 of the cDNA base sequence and cytosine C to thymine T at position 857 thereof, respectively, using:

(29 mer; SEQ ID NO: 10)

r856C857T-sen:
CGGTGCTGCTTCTTCTCACCGTCTTCCTG,
and

(29 mer; SEQ ID NO: 11)

r856C857T-ant:
CAGGAAGACGGTGAGAAGAAGCAGCACCG.

The base sequence of the resulting mutated cDNA is as shown in SEQ ID NO: 12. This allows a mutation of amino acid residue Ser to amino acid residue Leu at position p.286 homologous to the α4 subunit of the human neuronal nicotinic acetylcholinergic receptor gene.

A probe was prepared in substantially the same manner as in Example, which consists of a 118 bp nucleotide (SEQ ID NO:23) located at positions c.104-c.221 of the mutated cDNA.

The recombinants were created in substantially the same manner as in Example 1 using the mutated cDNA with the probe introduced therein as above.

### Example 3

In substantially the same manner as in Example 1, GCT was inserted between positions 878 and 879 of the cDNA base sequence, using the sense primer:

(30 mer; SEQ ID NO: 13)

rCHRNA-878insGCT-sen:
GTCTTCCTGCTGCTGCTCATCACCGAGATC,

and the antisense primer:

(30 mer; SEQ ID NO: 14)

rCHRNA-878insGCT-ant:
GATCTCGGTGATGAGCAGCAGCAGGAAGAC.

The base sequence of the resulting mutated cDNA is as shown in SEQ ID NO: 15. This mutation allows an insertopn of amino acid residue Leu between the amino acid residues Leu and Ile at positions p.293 and p.294, respectively, homologous to the α4 subunit of the human neuronal nicotinic acetylcholinergic receptor gene.

The mutated cDNA was then used to create recombinants in substantially the same manner as in Example 1.

### Example 4

The mutations c.856G>C and c.856G>A were introduced into the rat Chrnb2 isolated by PCR cloning, using a mutation introducing method. Specifically, PCR was performed using a primer set consisting of two primers designed based on a known cDNA sequence of rat Chrnb2 using Rat Brain QUICK-Clone cDNA (CLONTECH; Mountain View, Calif.) as a template, namely,

(41 mer, SEQ ID NO: 5)

BN-Rat CHRNB2 cDNA-F:
AGATCTCGCGACATGGCCGGGCACTCCAACTCAATGGCGCT,
and

(40 mer; SEQ ID NO: 6)

Rat CHRNB2 cDNA-CB-R:
ATCGATGGATCCTCACTTGGAGCTGGGAGCTGAGTGGTCA.

The PCR product was purified and subcloned into a pCRII-TOPO vector (Invitorogen; Carlsbad, Calif.). The resulting clone was sequenced to confirm the base sequence of the Chrnb2 cDNA. The rat base sequence information is available under accession number NM_{—}019297 (NCBI).

The mutation was introduced into the clone using QuikChange Site-Directed Mutagenesis Kit (STRATAGENE, La Jolla, Calif.). First, guanine G was mutated to cytosine C at position 856 of the cDNA base sequence using:

(30 mer; SEQ ID NO: 17)

Rat CHRNB2 m286V/M-F: CTCATCTCCAAGATTATGCCTCCCACCT
CC,
and

(30 mer; SEQ ID NO: 18)

Rat CHRNB2 m286V/M-R: GGAGGTGGGAGGCATAATCTTGGAGATG
AG.

This mutation results in a mutation of amino acid residue Val to amino acid residue Leu at position p.286 homologous to the β2 subunit of the human neuronal nicotinic acetylcholinergic receptor gene (β is used to comply with the on-line application regulations set by JPO).

The introduction of the mutation c.856G>C was arranged so as to cause a restriction enzyme site Hinf If to appear, thereby enabling an easy identification of the recombinants upon creating recombinants.

A probe (SEQ ID NO: 24) having a base sequence different from the original base sequence yet encoding the same amino acid sequence was introduced at the site of the mutated cDNA located from c.1171 to c.1232.

(SEQ ID NO: 24)

AACCCCGCCTCCGTCCAAGGACTCGCCGGCGCCTTTAGGGCCGAACCTAC
CGCCGCTGGCCC

After the 62-bp nucleotide was prepared and hybridized, it was inserted into a pCRII-TOPO vector using the restriction enzyme sites Hinc II and Sma I of the Chrnb2 cDNA inserted in the vector. The insert was sequenced in each step, and the base sequence was confirmed.

The two types of rat Chrnb2 cDNA with the mutation and the probe were transferred to an expression vector with a PDGF promoter derived from a pCI-neo vector, using the restriction enzyme sites Xho I and Sal I, and then cut with restriction enzymes SnaBI and DraIII. The fragment including the PDGF promoter, the rat Chrnb2 cDNA and a poly A portion was isolated by gel processing and purified. The isolated DNA was microinjected into fertilized eggs, and over 200 eggs in good condition were transplanted into the oviduct of recipient females. The animals were allowed to give natural birth to recombinants.

### Example 5

In substantially the same manner as in Example 4, guanine G was mutated to adenine A mutation at position 856 of the cDNA base sequence using:

(30 mer, SEQ ID NO: 20)

Rat CHRNB2 m286V/L-F: CTCATCTCCAAGATTCTGCCTCCCACCT
CC,
and

(30 mer; SEQ ID NO: 21)

Rat CHRNB2 m286V/L-R: GGAGGTGGGAGGCAGAATCTTGGAGATG
AG.

This mutation allows a replacement of amino acid residue Val by amino acid residue Met at position p.286 homologous to the β2 subunit of the human neuronal nicotinic acetylcholinergic receptor. The mutated cDNA was used to create recombinants in substantially the same manner as in Example 4.

### Example 6

### Epilepsy Model Rat Electroencephalography (EEG)

The transgenic rats (Chrnb2 V287L), 8 to 10 weeks of age, were subjected to electroencephalography (EEG). The rats were anesthetized with nembutal (Dainippon Sumitomo Pharma Co., Ltd.), and anchored to a brain stereotaxis apparatus (SR-5, Narishige). The electrodes were placed 2.5 mm (arterial) and 1.5 mm (lateral), and −2.5 mm (arterial) and 2.5 mm (lateral) from the bregma of the rat skull. The indifferent electrode was placed on a thick portion of the rat bone in the vicinity of the nasal bone. A dental cement was used to fix these electrodes. Teflon®-coated stainless steel electrodes for electroencephalography were placed on the frontal lobes of the left and right cerebral hemispheres (A=3. mm, L=0.8 mm from the bregma), and fixed with a dental cement. The indifferent electrode was implanted on the upper part of the cerebellum portion. The electroencephalogram (EEG) was taken with a VideoOption (Kissei Comtec) under freely moving conditions in a chamber over a time period of several days. Electroencephalogram analysis was made using a SleepSign (Kissei Comtec).

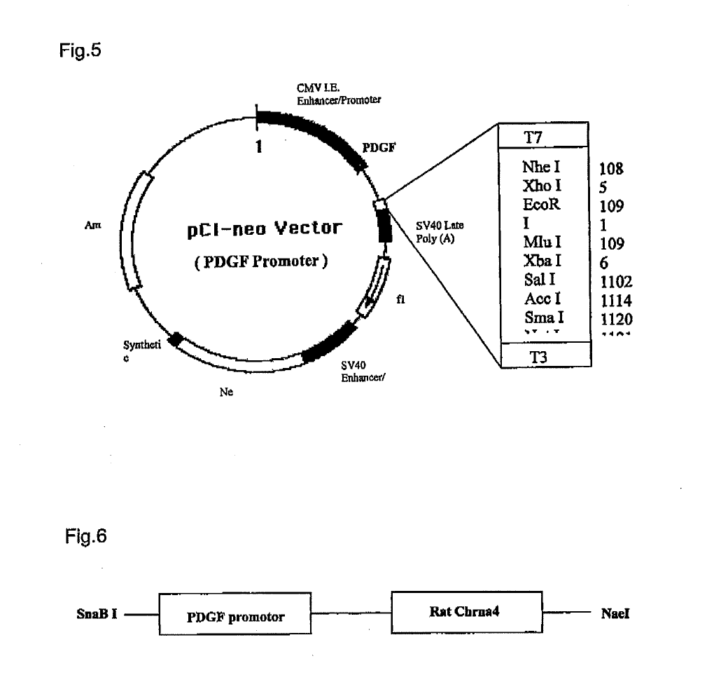

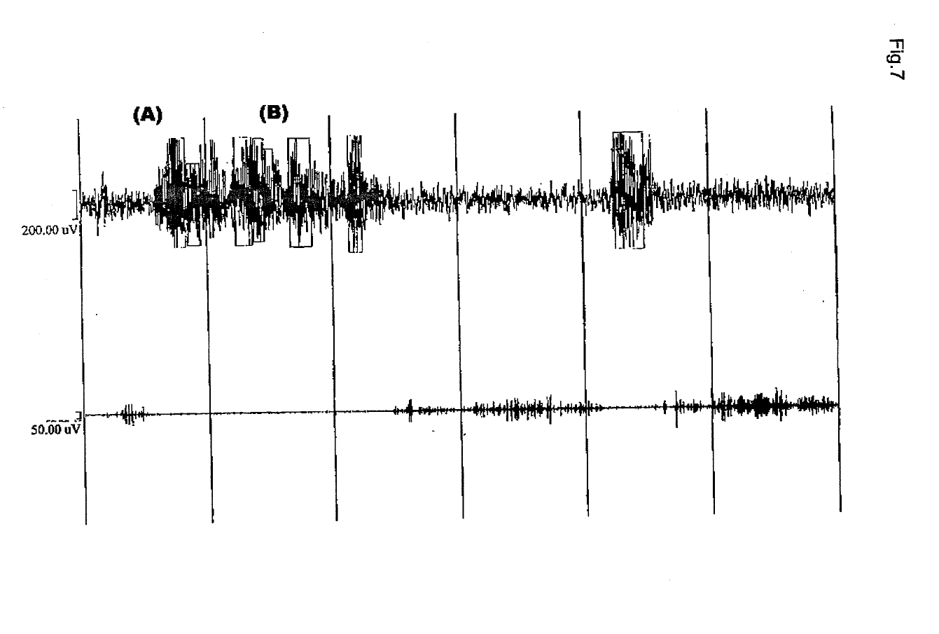

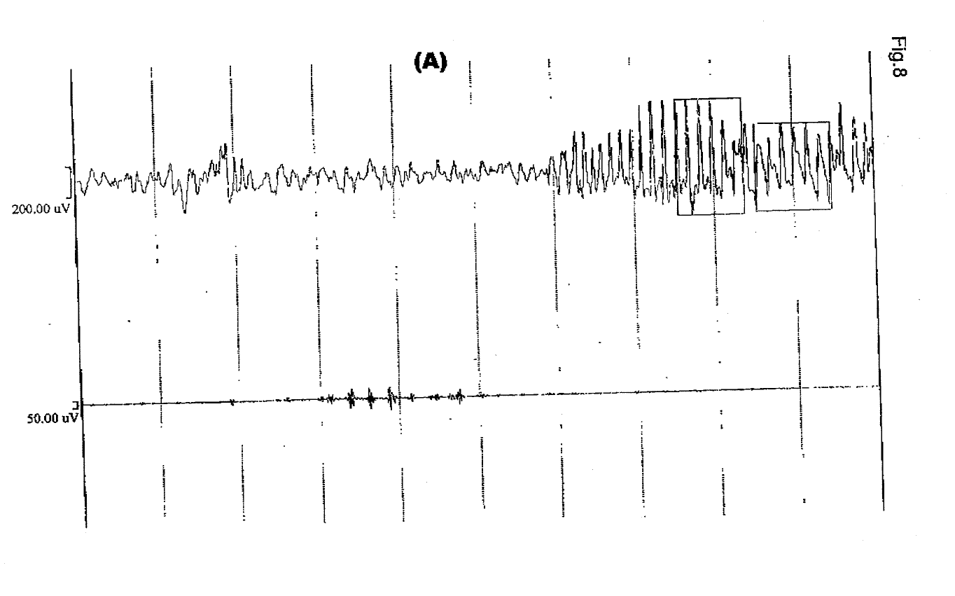

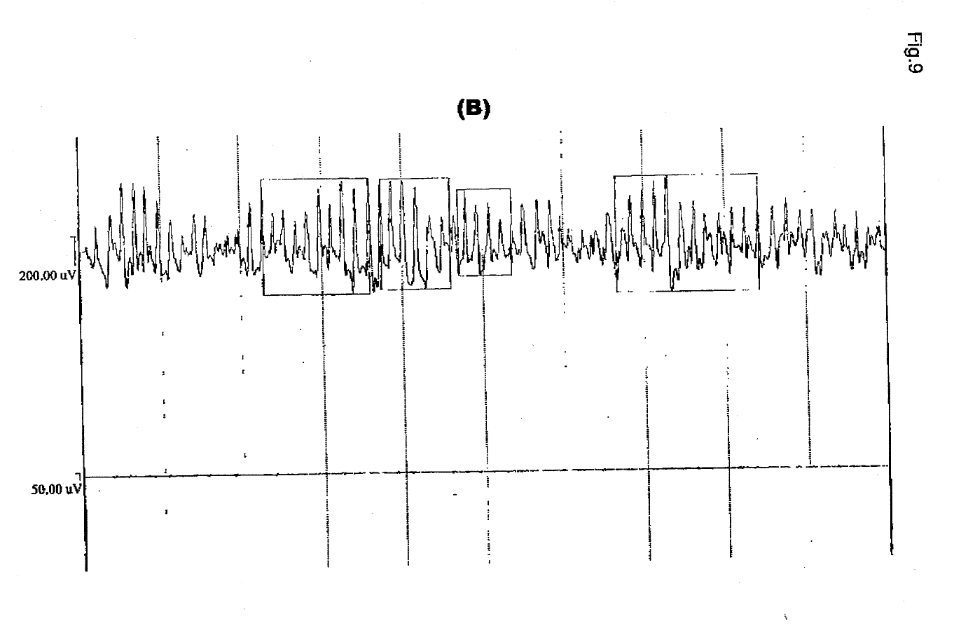

FIG. 5 represents the result of electroencephalography. In the figure, portions of abnormal brain waves are indicated by symbols A and B. FIG. 6 is a magnified view of the abnormal brain wave portion A. FIG. 7 shows the abnormal brain wave portion B. The results confirmed that the rat according to the present invention is usable as a model animal of epilepsy.

### INDUSTRIAL AVAILABILITY

A model non-human mammalian animal for human epilepsy according to the present invention has the same genetic defects as those in human epilepsy. The model animal can thus be used for the studies of epilepsy by inducing seizure attacks equivalent to human epileptic seizures. The model animal is therefore usable for the research and development of therapeutic drugs for epilepsy.

1-33. (canceled)

34. A model non-human mammalian animal for human epilepsy with genetic defects identical to genetic defects in human epilepsy, characterized in that the model non-human mammalian animal for human epilepsy possesses a mutated gene which is produced by introducing a genetic mutation to a non-human mammalian Chrna4 or Chrnb2 of the α4 subunit CHRNA4 or β2 subunit CHRNB2 of neuronal nicotinic acetylcholinergic receptor gene, respectively, associated with human autosomal dominant nocturnal frontal lobe epilepsy and which includes a probe having a base sequence different from the original base sequence yet coding for the same amino sequence.

35. The model non-human mammalian animal for human epilepsy as claimed in claim 34, wherein the mutation in the mutated gene possesses a new restriction site for a restriction enzyme.

36. The model non-human mammalian animal for human epilepsy as claimed in claim 34 or 35, wherein:
said Chrna4 is mutated to a base sequence of SEQ ID NO. 9 in which base C (cytosine) at position 845 of cDNA thereof is mutated to base T (thymine) (c.845C>T) and base G (guanine) at position 846 thereof is mutated to base C (cytosine) (c.846G>C); or
said Chrna4 is mutated to a base sequence of SEQ ID NO. 12 in which base T (thymine) at position 856 of cDNA thereof is mutated to base C (cytosine) (c.856T>C) and/or base C (cytosine) at position 857 thereof is mutated to base T (thymine) (c.857C>T); or
said Chrnb2 is mutated to a base sequence of SEQ ID NO. 19 or 22 in which base G (guanine) at position 856 of cDNA thereof is mutated to base C (cytosine) (c.856G>C) or to base A (adenine) (c.856G>A), respectively, or
codon GCT (c.879-880insGCT) or codon TTA (c.879-880insTTA) are inserted between positions 878-879 and 879-880 of the Chrna4 cDNA, respectively.

37. The model non-human mammalian animal for human epilepsy as claimed in claim 36, wherein:
amino acid residue Ser homologous to the acid residue at position p.282 of the α4 subunit CHRNA4 is replaced by amino acid residue Phe by mutation of c.845T>C and/or c.846G>A into cDNA of said Chrna4, respectively; or
amino acid residue Ser homologous to the acid residue at position p.286 of the α4 subunit CHRNA4 is replaced by amino acid residue Leu by mutation of c.856T>C and c.857C>T into said Chrna4; or
amino acid residue Val homologous to p.286 of the β2 subunit CHRNB2 is replaced by amino acid residue Leu or Met by mutation of c.856G>C or c.856G>A into said Chrnb2, respectively, or
amino acid residue Leu is inserted between amino acid residue Leu homologous to the amino acid residue at position p.293 of CHRNA4 and amino acid residue Ile homologous thereto at position p.194 by insertion of GCT (c.878-879insGCT) or TTA (c.879-880insTTA).

38. A method for the production of a model non-human mammalian animal for human epilepsy as claimed in claim 34 or 35, comprising:
preparing a mutated gene by inserting a genetic mutation in a Chrna4 or Chrnb2 cDNA of a non-human mammalian animal, said genetic mutation relating to genetic defects in α4 subunit CHRNA4 or β2 subunit CHRNB2 of neuronal nicotinic acetylcholinergic receptor gene associated with human autosomal dominant nocturnal frontal lobe epilepsy, and by replacing a part of the base sequence of the cDNA with a probe that has a base sequence different from said part of the cDNA base sequence yet codes for the same amino sequence;
transferring the mutated gene into an expression vector; and
transplanting the mutated gene in the expression vector into a recipient female via a fertilized egg to produce a recombinant non-human mammalian animal.

39. The method as claimed in claim 38, wherein the mutated gene is provided with a new restriction site for a restriction enzyme by mutation.

40. The method as claimed in claim 38, wherein:
base C (cytosine) at position 845 of Chrna4 cDNA is mutated to base T (thymine) (c.845 C>T) and/or base G (guanine) at position 846 of Chrna4 cDNA is mutated to base C (cytosine) (c.846G>C); or
base T (thymine) at position 856 of Chrna4 cDNA is mutated to base C (cytosine) (c.856T>C) and base C (cytosine) at position 857 of Chrna4 cDNA is mutated to base T (thymine) (c.857C>T); or
a codon GCT (c.879-880insGCT) is inserted between positions 878 and 879 or a codon TTA (c.879-880insTTA) is inserted between position 879 and 880 of Chrna4 cDNA, or
base G (guanine) at position p.856 of Chrnb2 cDNA is mutated to base C (cytosine) (c.856G>C) or to base A (adenine) (c.856G>A).

41. The method as claimed in claim 40, wherein:
amino acid residue Ser at position p.282 homologous to α4 subunit CHRNA4 is mutated to amino acid residue Phe or amino acid residue Ser at position p.284 thereof is mutated to amino acid residue Leu or amino acid residue Leu is inserted between amino acid residue Leu at position 293 thereof and amino acid residue Ile at position p.294 thereof, respectively, by mutation of c.845 C>T and c.846G>C or c.856T>C and c.857C>T or c.878-879insGCT or c.879-880insTTA in said Chrna4 cDNA, or
amino acid residue Val at position 286 homologous to β2 subunit CHRNB2 by mutation by insertion of c.856G>C or c.856G>A into the Chrnb2 is replaced by amino acid residue Leu or Met, respectively.

42. A mutated gene of a homologous gene Chrna4 or Chrnb2 of a non-human mammalian animal homologous to α4 subunit CHRNA4 or β2 subunit CHRNB2 of human neuronal nicotinic acetylcholinergic receptor gene, respectively, associated with human autosomal dominant nocturnal frontal lobe epilepsy,
comprising:
a genetic mutation introduced to the homologous gene Chrna4 or Chrnb2 homologous Chrna4 or Chrnb2 of the α4 subunit CHRNA4 or β2 subunit CHRNB2 of the human neuronal nicotinic acetylcholinergic receptor gene, respectively;
a probe having a base sequence that is different from a portion of the base sequence of the homologous gene yet codes for the same amino acid sequence as that of the portion of the base sequence thereof; and
a new restriction site for a restriction enzyme created by insertion of the genetic mutation.

43. The mutated gene as claimed in claim 42, wherein the mutated gene has:
base T (thymine) at position 845 of the Chrna4 cDNA mutated from base C (cytosine) (c.845C>T);
base C (cytosine) at position 846 of the Chrna4 cDNA mutated from base G (guanine) (c.846G>C);
base C (cytosine) at position p.856 of the Chrna4 cDNA mutated from base T (thymine) (c.856T>C);
base T (thymine) at position p.857 of the Chrna4 cDNA mutated from base C (cytosine) (c.857C>T); or
a codon GCT inserted between positions 878 and 879 of the Chrna4 cDNA (878-879insGCT) or a codon TTA inserted between positions 879 and 880 (879-880insTTA) thereof; or
base C (cystosine) or base A (adenine) mutated from base guanine at position 856 of the Chrnb2 cDNA is muted (c.856G>C) or (c.856G>A), respectively,
wherein:
amino acid residue Phe by which amino acid residue Ser at p.280 homologous to the α4 subunit CHRNA4 is replaced by mutation of c.845 C>T and c.846G>C to Chrna4 cDNA;
amino acid residue Leu by which amino acid residue Ser at p.284 homologous to the α4 subunit CHRNA4 is replaced by mutation of c.856T>C and c.857C>T to Chrna4 cDNA;
or amino acid residue Leu inserted between amino acid Leu at position p.293 homologous to the α4 subunit CHRNA4 and amino acid residue Ile at position p.294 homologous to the α4 subunit CHRNA4, respectively, by mutation of c.878-879insGCT or c.879-880insTTA to Chrna4 cDNA; or
amino acid residue Leu or Met, respectively, by which amino acid residue Val at p.286 homologous to the β2 subunit CHRNB2 is replaced by introduction of c.856G>C or c.856G>A into the Chrnb2.

44. A mutated gene of a homologous gene Chrna4 or Chrnb2 of a non-human mammalian animal homologous to α4 subunit CHRNA4 or β2 subunit CHRNB2 of human neuronal nicotinic acetylcholinergic receptor gene, respectively, associated with human autosomal dominant nocturnal frontal lobe epilepsy for producing the model non-human mammalian animal for human epilepsy as claimed in claim 34 or 35,
comprising:
a genetic mutation introduced to the homologous gene Chrna4 or Chrnb2 homologous Chrna4 or Chrnb2 of the α4 subunit CHRNA4 or β2 subunit CHRNB2 of the human neuronal nicotinic acetylcholinergic receptor gene, respectively;
a probe having a base sequence that is different from a portion of the base sequence of the homologous gene yet codes for the same amino acid sequence as that of the portion of the base sequence thereof; and
a new restriction site for a restriction enzyme created by insertion of the genetic mutation.

45. The mutated gene as claimed in claim 44, wherein the mutated gene has:
base T (thymine) at position 845 of the Chrna4 cDNA mutated from base C (cytosine) (c.845C>T);
base C (cytosine) at position 846 of the Chrna4 cDNA mutated from base G (guanine) (c.846G>C);
base C (cytosine) at position p.856 of the Chrna4 cDNA mutated from base T (thymine) (c.856T>C);
base T (thymine) at position p.857 of the Chrna4 cDNA mutated from base C (cytosine) (c.857C>T); or
a codon GCT inserted between positions 878 and 879 of the Chrna4 cDNA (878-879insGCT) or a codon TTA inserted between positions 879 and 880 (879-880insTTA) thereof; or
base C (cystosine) or base A (adenine) mutated from base guanine at position 856 of the Chrnb2 cDNA is muted (c.856G>C) or (c.856G>A), respectively,
wherein:
amino acid residue Phe by which amino acid residue Ser at p.280 homologous to the α4 subunit CHRNA4 is replaced by mutation of c.845 C>T and c.846G>C to Chrna4 cDNA;
amino acid residue Leu by which amino acid residue Ser at p.284 homologous to the α4 subunit CHRNA4 is replaced by mutation of c.856T>C and c.857C>T to Chrna4 cDNA;
or amino acid residue Leu inserted between amino acid Leu at position p.293 homologous to the α4 subunit CHRNA4 and amino acid residue Ile at position p.294 homologous to the α4 subunit CHRNA4, respectively, by mutation of c.878-879insGCT or c.879-880insTTA to Chrna4 cDNA; or
amino acid residue Leu or Met, respectively, by which amino acid residue Val at p.286 homologous to the β2 subunit CHRNB2 is replaced by introduction of c.856G>C or c.856G>A into the Chrnb2.

46. A method for identifying a homologous recombinant of the model non-human mammalian animal for human epilepsy as claimed in claim 34 or 35, comprising detecting a probe with a restriction enzyme, which is introduced into the homologous recombinant of a mutated gene, wherein the mutated gene is:
(i) a mutated gene of a homologous gene Chrna4 or Chrnb2 of a non-human mammalian animal homologous to α4 subunit CHRNA4 or β2 subunit CHRNB2 of human neuronal nicotinic acetylcholinergic receptor gene, respectively, associated with human autosomal dominant nocturnal frontal lobe epilepsy,
comprising:
a genetic mutation introduced to the homologous gene Chrna4 or Chrnb2 homologous Chrna4 or Chrnb2 of the α4 subunit CHRNA4 or β2 subunit CHRNB2 of the human neuronal nicotinic acetylcholinergic receptor gene, respectively;
a probe having a base sequence that is different from a portion of the base sequence of the homologous gene yet codes for the same amino acid sequence as that of the portion of the base sequence thereof; and
a new restriction site for a restriction enzyme created by insertion of the genetic mutation, or
(ii) a mutated gene of a homologous gene Chrna4 or Chrnb2 of a non-human mammalian animal homologous to α4 subunit CHRNA4 or β2 subunit CHRNB2 of human neuronal nicotinic acetylcholinergic receptor gene, respectively, associated with human autosomal dominant nocturnal frontal lobe epilepsy,
comprising:
a genetic mutation introduced to the homologous gene Chrna4 or Chrnb2 homologous Chrna4 or Chrnb2 of the α4 subunit CHRNA4 or β2 subunit CHRNB2 of the human neuronal nicotinic acetylcholinergic receptor gene, respectively;
a probe having a base sequence that is different from a portion of the base sequence of the homologous gene yet codes for the same amino acid sequence as that of the portion of the base sequence thereof; and
a new restriction site for a restriction enzyme created by insertion of the genetic mutation,
a probe having a base sequence which is different from a portion of a base sequence of a homologous gene Chrna4 or Chrnb2 of a non-human mammalian animal, respectively, homologous to α4 subunit CHRNA4 or β2 subunit CHRNB2 of human neuronal nicotinic acetylcholinergic receptor gene associated with human autosomal dominant noctural frontal lobe epilepsy yet which codes for the same amino acid sequence as the part of the base sequence.

47. The method as claimed in claim 46, wherein the homologous recombinant is identified by detecting the new restriction site for the restriction using the restriction enzyme, a new restriction site being created by mutation to the mutated gene.


SEQUENCE LISTING (31 sequences)

SEQ ID NO: 1 - DNA, 1890 bp, Rattus norvegicus
  misc_feature (1)..(1890): Rat Chrna4
atggccaattcgggcaccggggcgccgccgccgctgctgctactgccgctgctgctgctcctagggaccggcctcttgcctgctagcagccacatagagacccgggcccatgcggaggagcggctcctgaagagactcttctccggttacaacaagtggtctcggccagtagccaatatctcagatgtggtcctcgtccgctttggcttgtccattgctcagctcattgacgtggacgagaagaaccagatgatgacaaccaacgtgtgggtgaagcaggagtggcacgactacaagctgcgctgggaccctggtgactacgagaatgtcacctccatccgcatcccctctgaactcatctggaggcctgacatcgtcctctacaacaatgcggatggagactttgcagtcacccacctgaccaaggcccacctgttctatgacggaagggtgcagtggacacccccagccatctataagagctcctgcagcatcgacgtcaccttcttcccctttgaccagcagaactgtaccatgaagtttggatcctggacctacgacaaggccaagattgacttagtgagcatgcatagccgtgtggaccaactggacttctgggaaagtggggagtgggtcatcgtggatgctgtgggcacctacaacaccaggaagtacgagtgctgtgccgagatctatcctgacatcacctatgccttcatcatccgacggctgccgctattctacaccatcaacctcatcatcccgtgcctgctcatctcctgtctcaccgtgctggtcttctatctgccttcagagtgtggcgagaaggtcacactgtgcatctcggtgctgctttctctcaccgtcttcctgctgctcatcaccgagatcatcccgtccacctcgctggtcatcccgctcatcggcgagtacctcctcttcaccatgatcttcgtcaccctctccatcgtcatcacggtcttcgtgctcaatgtgcaccaccgctcgccacgcacacacacgatgcccgcctgggtgcgtagagtcttcctggacatcgtgcctcgcctcctcttcatgaagcgcccctctgtggtcaaagacaactgccggagacttattgagtccatgcacaagatggccaacgccccccgcttctggccagagcctgtgggcgagcccggcatcttgagtgacatctgcaaccaaggtctgtcacctgccccaactttctgcaaccccacggacacagcagtcgagacccagcctacgtgcaggtcacccccccttgaggtccctgacttgaagacatcagaggttgagaaggccagtccctgtccatcgcctggctcctgtcctccacccaagagcagcagtggggctccaatgctcatcaaagccaggtccctgagtgtccagcatgtgcccagctcccaagaagcagcagaagatggcatccgctgccggtctcggagtatccagtactgtgtttcccaagatggagctgcctccctggctgacagcaagcccaccagctccccgacctccctgaaggcccgtccatcccagcttcccgtgtcagaccaggcctctccatgcaaatgcacatgcaaggaaccatctcctgtgtccccagtcactgtgctcaaggcgggaggcaccaaagcacctccccaacacctgcccctgtcaccagccctgacacgggcagtagaaggcgtccagtacattgcagaccacctcaaggcagaagacactgacttctcggtgaaggaggactggaaatacgtggccatggtcattgaccgaatcttcctctggatgttcatcattgtctgccttctgggcactgtgggactcttcctgcctccctggctggctgcttgctga

SEQ ID NO: 2 - DNA, 1503 bp, Rattus norvegicus
  misc_feature (1)..(1503): Rat Chrnb2
atggccgggcactccaactcaatggcgctgttcagcttcagccttctttggctgtgctcaggggttttgggaactgacacagaggagcggctagtggagcatctcttagatccctcccgctataacaagctgattcgtccagctactaacggctctgagctggtgactgtacagctcatggtatcattggctcagctcattagtgtgcacgagcgggagcagatcatgaccaccaatgtctggctgacccaggagtgggaagattaccgcctcacatggaagcctgaggacttcgacaatatgaagaaagtccggctcccttccaaacacatctggctcccagatgtggttctatacaacaatgctgacggcatgtacgaagtctccttctattccaatgctgtggtctcctatgatggcagcatcttttggctaccacctgccatctacaagagtgcatgcaagattgaggtgaagcacttcccatttgaccagcagaattgcaccatgaagtttcgctcatggacctacgaccgtactgagattgacctggtgctcaaaagtgatgtggccagtctggatgacttcacacccagcggggagtgggacatcatcgcactgccaggccgacgcaacgagaacccagacgactccacctatgtggacatcacctatgacttcatcattcgtcgcaaaccactcttctacactatcaacctcatcatcccctgcgtactcatcacctcgctggccatcctggtcttctacctgccctcagactgtggtgaaaagatgacactttgtatttctgtgctgctagcactcacggtgttcctgctgctcatctccaagattgtgcctcccacctccctcgatgtaccgctggtgggcaagtacctcatgtttaccatggtgctagtcaccttctccatcgtcaccagcgtgtgtgtgctcaatgtgcaccaccgctcgcctaccacgcacaccatggccccctgggtcaaggtggtcttcctggagaagctgcccaccctgctcttcctgcagcagccacgccaccgctgtgcacgtcagcgtctgcgcttgaggaggcgccagcgagagcgtgagggcgcaggcgcgcttttcttccgtgaaggtcctgcggctgacccatgtacctgctttgtcaaccctgcatcagtgcagggcttggctggggctttccgagctgagcccactgcagccggcccggggcgctctgtggggccatgcagctgtggcctccgggaagcagtggatggcgtacgcttcattgcggaccacatgcgaagtgaggatgatgaccagagtgtgagggaggactggaaatacgttgccatggtgatcgaccgcctgttcctgtggatctttgtctttgtctgtgtctttgggaccgtcggcatgttcctgcagcctctcttccagaactacactgccactaccttcctccaccctgaccactcagctcccagctccaagtga

SEQ ID NO: 3 - DNA, 40 bp, Artificial sequence
  Synthetic construct; forward primer Chrna4
agatctcgcgaagcttcaccatggccaattcgggcaccgg

SEQ ID NO: 4 - DNA, 38 bp, Artificial sequence
  Synthetic construct; reverse primer Chrna4
agatctagatcagcaagcagccagccagggaggcagga

SEQ ID NO: 5 - DNA, 41 bp, Artificial sequence
  Synthetic construct; forward primer Chrnb2
agatctcgcgacatggccgggcactccaactcaatggcgct

SEQ ID NO: 6 - DNA, 40 bp, Artificial sequence
  Synthetic construct; reverse primer Chrnb2
atcgatggatcctcacttggagctgggagctgagtggtca

SEQ ID NO: 7 - DNA, 29 bp, Artificial sequence
  Synthetic construct; sense primer Chrna4 S282F
cacactgtgcatcttcgtgctgctttctc

SEQ ID NO: 8 - DNA, 29 bp, Artificial sequence
  Synthetic construct; antisense primer Chrna4 S282F
gagaaagcagcacgaagatgcacagtgtg

SEQ ID NO: 9 - DNA, 1889 bp, Rattus norvegicus
atggccaattcgggcaccggggcgccgccgccgctgctgctactgccgctgctgctgctcctagggaccggcctcttgcctgctagcagccacatagagacccggcccatgcggaggagcggctcctgaagagactcttctccggttacaacaagtggtctcggccagtagccaatatctcagatgtggtcctcgtccgctttggcttgtccattgctcagctcattgacgtggacgagaagaaccagatgatgacaaccaacgtgtgggtgaagcaggagtggcacgactacaagctgcgctgggaccctggtgactacgagaatgtcacctccatccgcatcccctctgaactcatctggaggcctgacatcgtcctctacaacaatgcggatggagactttgcagtcacccacctgaccaaggcccacctgttctatgacggaagggtgcagtggacacccccagccatctataagagctcctgcagcatcgacgtcaccttcttcccctttgaccagcagaactgtaccatgaagtttggatcctggacctacgacaaggccaagattgacttagtgagcatgcatagccgtgtggaccaactggacttctgggaaagtggggagtgggtcatcgtggatgctgtgggcacctacaacaccaggaagtacgagtgctgtgccgagatctatcctgacatcacctatgccttcatcatccgacggctgccgctattctacaccatcaacctcatcatcccgtgcctgctcatctcctgtctcaccgtgctggtcttctatctgccttcagagtgtggcgagaaggtcacactgtgcatcttcgtgctgctttctctcaccgtcttcctgctgctcatcaccgagatcatcccgtccacctcgctggtcatcccgctcatcggcgagtacctcctcttcaccatgatcttcgtcaccctctccatcgtcatcacggtcttcgtgctcaatgtgcaccaccgctcgccacgcacacacacgatgcccgcctgggtgcgtagagtcttcctggacatcgtgcctcgcctcctcttcatgaagcgcccctctgtggtcaaagacaactgccggagacttattgagtccatgcacaagatggccaacgccccccgcttctggccagagcctgtgggcgagcccggcatcttgagtgacatctgcaaccaaggtctgtcacctgccccaactttctgcaaccccacggacacagcagtcgagacccagcctacgtgcaggtcacccccccttgaggtccctgacttgaagacatcagaggttgagaaggccagtccctgtccatcgcctggctcctgtcctccacccaagagcagcagtggggctccaatgctcatcaaagccaggtccctgagtgtccagcatgtgcccagctcccaagaagcagcagaagatggcatccgctgccggtctcggagtatccagtactgtgtttcccaagatggagctgcctccctggctgacagcaagcccaccagctccccgacctccctgaaggcccgtccatcccagcttcccgtgtcagaccaggcctctccatgcaaatgcacatgcaaggaaccatctcctgtgtccccagtcactgtgctcaaggcgggaggcaccaaagcacctccccaacacctgcccctgtcaccagccctgacacgggcagtagaaggcgtccagtacattgcagaccacctcaaggcagaagacactgacttctcggtgaaggaggactggaaatacgtggccatggtcattgaccgaatcttcctctggatgttcatcattgtctgccttctgggcactgtgggactcttcctgcctccctggctggctgcttgctga

SEQ ID NO: 10 - DNA, 29 bp, Artificial sequence
  Synthetic construct; sense primer Chrna4 S286L
cggtgctgcttcttctcaccgtcttcctg

SEQ ID NO: 11 - DNA, 29 bp, Artificial sequence
  Synthetic construct; antisense primer Chrna4 S286L
caggaagacggtgagaagaagcagcaccg

SEQ ID NO: 12 - DNA, 1890 bp, Rattus norvegicus
atggccaattcgggcaccggggcgccgccgccgctgctgctactgccgctgctgctgctcctagggaccggcctcttgcctgctagcagccacatagagacccgggcccatgcggaggagcggctcctgaagagactcttctccggttacaacaagtggtctcggccagtagccaatatctcagatgtggtcctcgtccgctttggcttgtccattgctcagctcattgacgtggacgagaagaaccagatgatgacaaccaacgtgtgggtgaagcaggagtggcacgactacaagctgcgctgggaccctggtgactacgagaatgtcacctccatccgcatcccctctgaactcatctggaggcctgacatcgtcctctacaacaatgcggatggagactttgcagtcacccacctgaccaaggcccacctgttctatgacggaagggtgcagtggacacccccagccatctataagagctcctgcagcatcgacgtcaccttcttcccctttgaccagcagaactgtaccatgaagtttggatcctggacctacgacaaggccaagattgacttagtgagcatgcatagccgtgtggaccaactggacttctgggaaagtggggagtgggtcatcgtggatgctgtgggcacctacaacaccaggaagtacgagtgctgtgccgagatctatcctgacatcacctatgccttcatcatccgacggctgccgctattctacaccatcaacctcatcatcccgtgcctgctcatctcctgtctcaccgtgctggtcttctatctgccttcagagtgtggcgagaaggtcacactgtgcatctcggtgctgcttcttctcaccgtcttcctgctgctcatcaccgagatcatcccgtccacctcgctggtcatcccgctcatcggcgagtacctcctcttcaccatgatcttcgtcaccctctccatcgtcatcacggtcttcgtgctcaatgtgcaccaccgctcgccacgcacacacacgatgcccgcctgggtgcgtagagtcttcctggacatcgtgcctcgcctcctcttcatgaagcgcccctctgtggtcaaagacaactgccggagacttattgagtccatgcacaagatggccaacgccccccgcttctggccagagcctgtgggcgagcccggcatcttgagtgacatctgcaaccaaggtctgtcacctgccccaactttctgcaaccccacggacacagcagtcgagacccagcctacgtgcaggtcacccccccttgaggtccctgacttgaagacatcagaggttgagaaggccagtccctgtccatcgcctggctcctgtcctccacccaagagcagcagtggggctccaatgctcatcaaagccaggtccctgagtgtccagcatgtgcccagctcccaagaagcagcagaagatggcatccgctgccggtctcggagtatccagtactgtgtttcccaagatggagctgcctccctggctgacagcaagcccaccagctccccgacctccctgaaggcccgtccatcccagcttcccgtgtcagaccaggcctctccatgcaaatgcacatgcaaggaaccatctcctgtgtccccagtcactgtgctcaaggcgggaggcaccaaagcacctccccaacacctgcccctgtcaccagccctgacacgggcagtagaaggcgtccagtacattgcagaccacctcaaggcagaagacactgacttctcggtgaaggaggactggaaatacgtggccatggtcattgaccgaatcttcctctggatgttcatcattgtctgccttctgggcactgtgggactcttcctgcctccctggctggctgcttgctga

SEQ ID NO: 13 - DNA, 30 bp, Artificial sequence
  Synthetic construct; sense primer Chrna4 c878-879insGCT
gtcttcctgctgctgctcatcaccgagatc

SEQ ID NO: 14 - DNA, 30 bp, Artificial sequence
  Synthetic construct; antisense primer Chrna4 c.878-879insGCT
gatctcggtgatgagcagcagcaggaagac

SEQ ID NO: 15 - DNA, 1893 bp, Rattus norvegicus
atggccaattcgggcaccggggcgccgccgccgctgctgctactgccgctgctgctgctcctagggaccggcctcttgcctgctagcagccacatagagacccgggcccatgcggaggagcggctcctgaagagactcttctccggttacaacaagtggtctcggccagtagccaatatctcagatgtggtcctcgtccgctttggcttgtccattgctcagctcattgacgtggacgagaagaaccagatgatgacaaccaacgtgtgggtgaagcaggagtggcacgactacaagctgcgctgggaccctggtgactacgagaatgtcacctccatccgcatcccctctgaactcatctggaggcctgacatcgtcctctacaacaatgcggatggagactttgcagtcacccacctgaccaaggcccacctgttctatgacggaagggtgcagtggacacccccagccatctataagagctcctgcagcatcgacgtcaccttcttcccctttgaccagcagaactgtaccatgaagtttggatcctggacctacgacaaggccaagattgacttagtgagcatgcatagccgtgtggaccaactggacttctgggaaagtggggagtgggtcatcgtggatgctgtgggcacctacaacaccaggaagtacgagtgctgtgccgagatctatcctgacatcacctatgccttcatcatccgacggctgccgctattctacaccatcaacctcatcatcccgtgcctgctcatctcctgtctcaccgtgctggtcttctatctgccttcagagtgtggcgagaaggtcacactgtgcatctcggtgctgctttctctcaccgtcttcctgctgctgctaatcaccgagatcatcccgtccacctcgctggtcatcccgctcatcggcgagtacctcctcttcaccatgatcttcgtcaccctctccatcgtcatcacggtcttcgtgctcaatgtgcaccaccgctcgccacgcacacacacgatgcccgcctgggtgcgtagagtcttcctggacatcgtgcctcgcctcctcttcatgaagcgcccctctgtggtcaaagacaactgccggagacttattgagtccatgcacaagatggccaacgccccccgcttctggccagagcctgtgggcgagcccggcatcttgagtgacatctgcaaccaaggtctgtcacctgccccaactttctgcaaccccacggacacagcagtcgagacccagcctacgtgcaggtcacccccccttgaggtccctgacttgaagacatcagaggttgagaaggccagtccctgtccatcgcctggctcctgtcctccacccaagagcagcagtggggctccaatgctcatcaaagccaggtccctgagtgtccagcatgtgcccagctcccaagaagcagcagaagatggcatccgctgccggtctcggagtatccagtactgtgtttcccaagatggagctgcctccctggctgacagcaagcccaccagctccccgacctccctgaaggcccgtccatcccagcttcccgtgtcagaccaggcctctccatgcaaatgcacatgcaaggaaccatctcctgtgtccccagtcactgtgctcaaggcgggaggcaccaaagcacctccccaacacctgcccctgtcaccagccctgacacgggcagtagaaggcgtccagtacattgcagaccacctcaaggcagaagacactgacttctcggtgaaggaggactggaaatacgtggccatggtcattgaccgaatcttcctctggatgttcatcattgtctgccttctgggcactgtgggactcttcctgcctccctggctggctgcttgctga

SEQ ID NO: 16 - DNA, 1893 bp, Rattus norvegicus
atggccaattcgggcaccggggcgccgccgccgctgctgctactgccgctgctgctgctcctagggaccggcctcttgcctgctagcagccacatagagacccgggcccatgcggaggagcggctcctgaagagactcttctccggttacaacaagtggtctcggccagtagccaatatctcagatgtggtcctcgtccgctttggcttgtccattgctcagctcattgacgtggacgagaagaaccagatgatgacaaccaacgtgtgggtgaagcaggagtggcacgactacaagctgcgctgggaccctggtgactacgagaatgtcacctccatccgcatcccctctgaactcatctggaggcctgacatcgtcctctacaacaatgcggatggagactttgcagtcacccacctgaccaaggcccacctgttctatgacggaagggtgcagtggacacccccagccatctataagagctcctgcagcatcgacgtcaccttcttcccctttgaccagcagaactgtaccatgaagtttggatcctggacctacgacaaggccaagattgacttagtgagcatgcatagccgtgtggaccaactggacttctgggaaagtggggagtgggtcatcgtggatgctgtgggcacctacaacaccaggaagtacgagtgctgtgccgagatctatcctgacatcacctatgccttcatcatccgacggctgccgctattctacaccatcaacctcatcatcccgtgcctgctcatctcctgtctcaccgtgctggtcttctatctgccttcagagtgtggcgagaaggtcacactgtgcatctcggtgctgctttctctcaccgtcttcctgctgctcttaatcaccgagatcatcccgtccacctcgctggtcatcccgctcatcggcgagtacctcctcttcaccatgatcttcgtcaccctctccatcgtcatcacggtcttcgtgctcaatgtgcaccaccgctcgccacgcacacacacgatgcccgcctgggtgcgtagagtcttcctggacatcgtgcctcgcctcctcttcatgaagcgcccctctgtggtcaaagacaactgccggagacttattgagtccatgcacaagatggccaacgccccccgcttctggccagagcctgtgggcgagcccggcatcttgagtgacatctgcaaccaaggtctgtcacctgccccaactttctgcaaccccacggacacagcagtcgagacccagcctacgtgcaggtcacccccccttgaggtccctgacttgaagacatcagaggttgagaaggccagtccctgtccatcgcctggctcctgtcctccacccaagagcagcagtggggctccaatgctcatcaaagccaggtccctgagtgtccagcatgtgcccagctcccaagaagcagcagaagatggcatccgctgccggtctcggagtatccagtactgtgtttcccaagatggagctgcctccctggctgacagcaagcccaccagctccccgacctccctgaaggcccgtccatcccagcttcccgtgtcagaccaggcctctccatgcaaatgcacatgcaaggaaccatctcctgtgtccccagtcactgtgctcaaggcgggaggcaccaaagcacctccccaacacctgcccctgtcaccagccctgacacgggcagtagaaggcgtccagtacattgcagaccacctcaaggcagaagacactgacttctcggtgaaggaggactggaaatacgtggccatggtcattgaccgaatcttcctctggatgttcatcattgtctgccttctgggcactgtgggactcttcctgcctccctggctggctgcttgctga

SEQ ID NO: 17 - DNA, 30 bp, Artificial sequence
  Synthetic construct; sense primer Chrnb2 V287L
ctcatctccaagattatgcctcccacctcc

SEQ ID NO: 18 - DNA, 30 bp, Artificial sequence
  Synthetic construct; antisense primer Chrnb2 S287L
ggaggtgggaggcataatcttggagatgag

SEQ ID NO: 19 - DNA, 1501 bp, Rattus norvegicus
ggccgggcactccaactcaatggcgctgttcagcttcagccttctttggctgtgctcaggggttttgggaactgacacagaggagcggctagtggagcatctcttagatccctcccgctataacaagctgattcgtccagctactaacggctctgagctggtgactgtacagctcatggtatcattggctcagctcattagtgtgcacgagcgggagcagatcatgaccaccaatgtctggctgacccaggagtgggaagattaccgcctcacatggaagcctgaggacttcgacaatatgaagaaagtccggctcccttccaaacacatctggctcccagatgtggttctatacaacaatgctgacggcatgtacgaagtctccttctattccaatgctgtggtctcctatgatggcagcatcttttggctaccacctgccatctacaagagtgcatgcaagattgaggtgaagcacttcccatttgaccagcagaattgcaccatgaagtttcgctcatggacctacgaccgtactgagattgacctggtgctcaaaagtgatgtggccagtctggatgacttcacacccagcggggagtgggacatcatcgcactgccaggccgacgcaacgagaacccagacgactccacctatgtggacatcacctatgacttcatcattcgtcgcaaaccactcttctacactatcaacctcatcatcccctgcgtactcatcacctcgctggccatcctggtcttctacctgccctcagactgtggtgaaaagatgacactttgtatttctgtgctgctagcactcacggtgttcctgctgctcatctccaagattctgcctcccacctccctcgatgtaccgctggtgggcaagtacctcatgtttaccatggtgctagtcaccttctccatcgtcaccagcgtgtgtgtgctcaatgtgcaccaccgctcgcctaccacgcacaccatggccccctgggtcaaggtggtcttcctggagaagctgcccaccctgctcttcctgcagcagccacgccaccgctgtgcacgtcagcgtctgcgcttgaggaggcgccagcgagagcgtgagggcgcaggcgcgcttttcttccgtgaaggtcctgcggctgacccatgtacctgctttgtcaaccctgcatcagtgcagggcttggctggggctttccgagctgagcccactgcagccggcccggggcgctctgtggggccatgcagctgtggcctccgggaagcagtggatggcgtacgcttcattgcggaccacatgcgaagtgaggatgatgaccagagtgtgagggaggactggaaatacgttgccatggtgatcgaccgcctgttcctgtggatctttgtctttgtctgtgtctttgggaccgtcggcatgttcctgcagcctctcttccagaactacactgccactaccttcctccaccctgaccactcagctcccagctccaagtga

SEQ ID NO: 20 - DNA, 30 bp, Artificial sequence
  Synthetic construct; sense primer Chrnb2 V287M
ctcatctccaagattctgcctcccacctcc

SEQ ID NO: 21 - DNA, 30 bp, Artificial sequence
  Synthetic construct; antisense primer Chrnb2 S287M
ggaggtgggaggcagaatcttggagatgag

SEQ ID NO: 22 - DNA, 1502 bp, Rattus norvegicus
tggccgggcactccaactcaatggcgctgttcagcttcagccttctttggctgtgctcaggggttttgggaactgacacagaggagcggctagtggagcatctcttagatccctcccgctataacaagctgattcgtccagctactaacggctctgagctggtgactgtacagctcatggtatcattggctcagctcattagtgtgcacgagcgggagcagatcatgaccaccaatgtctggctgacccaggagtgggaagattaccgcctcacatggaagcctgaggacttcgacaatatgaagaaagtccggctcccttccaaacacatctggctcccagatgtggttctatacaacaatgctgacggcatgtacgaagtctccttctattccaatgctgtggtctcctatgatggcagcatcttttggctaccacctgccatctacaagagtgcatgcaagattgaggtgaagcacttcccatttgaccagcagaattgcaccatgaagtttcgctcatggacctacgaccgtactgagattgacctggtgctcaaaagtgatgtggccagtctggatgacttcacacccagcggggagtgggacatcatcgcactgccaggccgacgcaacgagaacccagacgactccacctatgtggacatcacctatgacttcatcattcgtcgcaaaccactcttctacactatcaacctcatcatcccctgcgtactcatcacctcgctggccatcctggtcttctacctgccctcagactgtggtgaaaagatgacactttgtatttctgtgctgctagcactcacggtgttcctgctgctcatctccaagattatgcctcccacctccctcgatgtaccgctggtgggcaagtacctcatgtttaccatggtgctagtcaccttctccatcgtcaccagcgtgtgtgtgctcaatgtgcaccaccgctcgcctaccacgcacaccatggccccctgggtcaaggtggtcttcctggagaagctgcccaccctgctcttcctgcagcagccacgccaccgctgtgcacgtcagcgtctgcgcttgaggaggcgccagcgagagcgtgagggcgcaggcgcgcttttcttccgtgaaggtcctgcggctgacccatgtacctgctttgtcaaccctgcatcagtgcagggcttggctggggctttccgagctgagcccactgcagccggcccggggcgctctgtggggccatgcagctgtggcctccgggaagcagtggatggcgtacgcttcattgcggaccacatgcgaagtgaggatgatgaccagagtgtgagggaggactggaaatacgttgccatggtgatcgaccgcctgttcctgtggatctttgtctttgtctgtgtctttgggaccgtcggcatgttcctgcagcctctcttccagaactacactgccactaccttcctccaccctgaccactcagctcccagctccaagtga

SEQ ID NO: 23 - DNA, 118 bp, Artificial sequence
  Synthetic construct; probe Chrna4
gggctcacgccgaagaacgcctgctcaaaaggctgttttctggctataataaatggtcccgccccgtggctaacatttccgacgtcgtgctggtgcggttcggattatctatcgctca

SEQ ID NO: 24 - DNA, 62 bp, Artificial sequence
  Synthetic construct; probe Chrnb2
aaccccgcctccgtccaaggactcgccggcgcctttagggccgaacctaccgccgctggccc

SEQ ID NO: 25 - DNA, 1890 bp, Rattus norvegicus
atggccaattcgggcaccggggcgccgccgccgctgctgctactgccgctgctgctgctcctagggaccggcctcttgcctgctagcagccacatagagacccgggctcacgccgaagaacgcctgctcaaaaggctgttttctggctataataaatggtcccgccccgtggctaacatttccgacgtcgtgctggtgcggttcggattatctatcgctcagctcattgacgtggacgagaagaaccagatgatgacaaccaacgtgtgggtgaagcaggagtggcacgactacaagctgcgctgggaccctggtgactacgagaatgtcacctccatccgcatcccctctgaactcatctggaggcctgacatcgtcctctacaacaatgcggatggagactttgcagtcacccacctgaccaaggcccacctgttctatgacggaagggtgcagtggacacccccagccatctataagagctcctgcagcatcgacgtcaccttcttcccctttgaccagcagaactgtaccatgaagtttggatcctggacctacgacaaggccaagattgacttagtgagcatgcatagccgtgtggaccaactggacttctgggaaagtggggagtgggtcatcgtggatgctgtgggcacctacaacaccaggaagtacgagtgctgtgccgagatctatcctgacatcacctatgccttcatcatccgacggctgccgctattctacaccatcaacctcatcatcccgtgcctgctcatctcctgtctcaccgtgctggtcttctatctgccttcagagtgtggcgagaaggtcacactgtgcatcttcgtgctgctttctctcaccgtcttcctgctgctcatcaccgagatcatcccgtccacctcgctggtcatcccgctcatcggcgagtacctcctcttcaccatgatcttcgtcaccctctccatcgtcatcacggtcttcgtgctcaatgtgcaccaccgctcgccacgcacacacacgatgcccgcctgggtgcgtagagtcttcctggacatcgtgcctcgcctcctcttcatgaagcgcccctctgtggtcaaagacaactgccggagacttattgagtccatgcacaagatggccaacgccccccgcttctggccagagcctgtgggcgagcccggcatcttgagtgacatctgcaaccaaggtctgtcacctgccccaactttctgcaaccccacggacacagcagtcgagacccagcctacgtgcaggtcacccccccttgaggtccctgacttgaagacatcagaggttgagaaggccagtccctgtccatcgcctggctcctgtcctccacccaagagcagcagtggggctccaatgctcatcaaagccaggtccctgagtgtccagcatgtgcccagctcccaagaagcagcagaagatggcatccgctgccggtctcggagtatccagtactgtgtttcccaagatggagctgcctccctggctgacagcaagcccaccagctccccgacctccctgaaggcccgtccatcccagcttcccgtgtcagaccaggcctctccatgcaaatgcacatgcaaggaaccatctcctgtgtccccagtcactgtgctcaaggcgggaggcaccaaagcacctccccaacacctgcccctgtcaccagccctgacacgggcagtagaaggcgtccagtacattgcagaccacctcaaggcagaagacactgacttctcggtgaaggaggactggaaatacgtggccatggtcattgaccgaatcttcctctggatgttcatcattgtctgccttctgggcactgtgggactcttcctgcctccctggctggctgcttgctga

SEQ ID NO: 26 - DNA, 1890 bp, Rattus norvegicus
atggccaattcgggcaccggggcgccgccgccgctgctgctactgccgctgctgctgctcctagggaccggcctcttgcctgctagcagccacatagagacccgggctcacgccgaagaacgcctgctcaaaaggctgttttctggctataataaatggtcccgccccgtggctaacatttccgacgtcgtgctggtgcggttcggattatctatcgctcagctcattgacgtggacgagaagaaccagatgatgacaaccaacgtgtgggtgaagcaggagtggcacgactacaagctgcgctgggaccctggtgactacgagaatgtcacctccatccgcatcccctctgaactcatctggaggcctgacatcgtcctctacaacaatgcggatggagactttgcagtcacccacctgaccaaggcccacctgttctatgacggaagggtgcagtggacacccccagccatctataagagctcctgcagcatcgacgtcaccttcttcccctttgaccagcagaactgtaccatgaagtttggatcctggacctacgacaaggccaagattgacttagtgagcatgcatagccgtgtggaccaactggacttctgggaaagtggggagtgggtcatcgtggatgctgtgggcacctacaacaccaggaagtacgagtgctgtgccgagatctatcctgacatcacctatgccttcatcatccgacggctgccgctattctacaccatcaacctcatcatcccgtgcctgctcatctcctgtctcaccgtgctggtcttctatctgccttcagagtgtggcgagaaggtcacactgtgcatctcggtgctgcttcttctcaccgtcttcctgctgctcatcaccgagatcatcccgtccacctcgctggtcatcccgctcatcggcgagtacctcctcttcaccatgatcttcgtcaccctctccatcgtcatcacggtcttcgtgctcaatgtgcaccaccgctcgccacgcacacacacgatgcccgcctgggtgcgtagagtcttcctggacatcgtgcctcgcctcctcttcatgaagcgcccctctgtggtcaaagacaactgccggagacttattgagtccatgcacaagatggccaacgccccccgcttctggccagagcctgtgggcgagcccggcatcttgagtgacatctgcaaccaaggtctgtcacctgccccaactttctgcaaccccacggacacagcagtcgagacccagcctacgtgcaggtcacccccccttgaggtccctgacttgaagacatcagaggttgagaaggccagtccctgtccatcgcctggctcctgtcctccacccaagagcagcagtggggctccaatgctcatcaaagccaggtccctgagtgtccagcatgtgcccagctcccaagaagcagcagaagatggcatccgctgccggtctcggagtatccagtactgtgtttcccaagatggagctgcctccctggctgacagcaagcccaccagctccccgacctccctgaaggcccgtccatcccagcttcccgtgtcagaccaggcctctccatgcaaatgcacatgcaaggaaccatctcctgtgtccccagtcactgtgctcaaggcgggaggcaccaaagcacctccccaacacctgcccctgtcaccagccctgacacgggcagtagaaggcgtccagtacattgcagaccacctcaaggcagaagacactgacttctcggtgaaggaggactggaaatacgtggccatggtcattgaccgaatcttcctctggatgttcatcattgtctgccttctgggcactgtgggactcttcctgcctccctggctggctgcttgctga

SEQ ID NO: 27 - DNA, 1893 bp, Rattus norvegicus
atggccaattcgggcaccggggcgccgccgccgctgctgctactgccgctgctgctgctcctagggaccggcctcttgcctgctagcagccacatagagacccgggctcacgccgaagaacgcctgctcaaaaggctgttttctggctataataaatggtcccgccccgtggctaacatttccgacgtcgtgctggtgcggttcggattatctatcgctcagctcattgacgtggacgagaagaaccagatgatgacaaccaacgtgtgggtgaagcaggagtggcacgactacaagctgcgctgggaccctggtgactacgagaatgtcacctccatccgcatcccctctgaactcatctggaggcctgacatcgtcctctacaacaatgcggatggagactttgcagtcacccacctgaccaaggcccacctgttctatgacggaagggtgcagtggacacccccagccatctataagagctcctgcagcatcgacgtcaccttcttcccctttgaccagcagaactgtaccatgaagtttggatcctggacctacgacaaggccaagattgacttagtgagcatgcatagccgtgtggaccaactggacttctgggaaagtggggagtgggtcatcgtggatgctgtgggcacctacaacaccaggaagtacgagtgctgtgccgagatctatcctgacatcacctatgccttcatcatccgacggctgccgctattctacaccatcaacctcatcatcccgtgcctgctcatctcctgtctcaccgtgctggtcttctatctgccttcagagtgtggcgagaaggtcacactgtgcatctcggtgctgctttctctcaccgtcttcctgctgctgctaatcaccgagatcatcccgtccacctcgctggtcatcccgctcatcggcgagtacctcctcttcaccatgatcttcgtcaccctctccatcgtcatcacggtcttcgtgctcaatgtgcaccaccgctcgccacgcacacacacgatgcccgcctgggtgcgtagagtcttcctggacatcgtgcctcgcctcctcttcatgaagcgcccctctgtggtcaaagacaactgccggagacttattgagtccatgcacaagatggccaacgccccccgcttctggccagagcctgtgggcgagcccggcatcttgagtgacatctgcaaccaaggtctgtcacctgccccaactttctgcaaccccacggacacagcagtcgagacccagcctacgtgcaggtcacccccccttgaggtccctgacttgaagacatcagaggttgagaaggccagtccctgtccatcgcctggctcctgtcctccacccaagagcagcagtggggctccaatgctcatcaaagccaggtccctgagtgtccagcatgtgcccagctcccaagaagcagcagaagatggcatccgctgccggtctcggagtatccagtactgtgtttcccaagatggagctgcctccctggctgacagcaagcccaccagctccccgacctccctgaaggcccgtccatcccagcttcccgtgtcagaccaggcctctccatgcaaatgcacatgcaaggaaccatctcctgtgtccccagtcactgtgctcaaggcgggaggcaccaaagcacctccccaacacctgcccctgtcaccagccctgacacgggcagtagaaggcgtccagtacattgcagaccacctcaaggcagaagacactgacttctcggtgaaggaggactggaaatacgtggccatggtcattgaccgaatcttcctctggatgttcatcattgtctgccttctgggcactgtgggactcttcctgcctccctggctggctgcttgctga

SEQ ID NO: 28 - DNA, 1501 bp, Rattus norvegicus
ggccgggcactccaactcaatggcgctgttcagcttcagccttctttggctgtgctcaggggttttgggaactgacacagaggagcggctagtggagcatctcttagatccctcccgctataacaagctgattcgtccagctactaacggctctgagctggtgactgtacagctcatggtatcattggctcagctcattagtgtgcacgagcgggagcagatcatgaccaccaatgtctggctgacccaggagtgggaagattaccgcctcacatggaagcctgaggacttcgacaatatgaagaaagtccggctcccttccaaacacatctggctcccagatgtggttctatacaacaatgctgacggcatgtacgaagtctccttctattccaatgctgtggtctcctatgatggcagcatcttttggctaccacctgccatctacaagagtgcatgcaagattgaggtgaagcacttcccatttgaccagcagaattgcaccatgaagtttcgctcatggacctacgaccgtactgagattgacctggtgctcaaaagtgatgtggccagtctggatgacttcacacccagcggggagtgggacatcatcgcactgccaggccgacgcaacgagaacccagacgactccacctatgtggacatcacctatgacttcatcattcgtcgcaaaccactcttctacactatcaacctcatcatcccctgcgtactcatcacctcgctggccatcctggtcttctacctgccctcagactgtggtgaaaagatgacactttgtatttctgtgctgctagcactcacggtgttcctgctgctcatctccaagattctgcctcccacctccctcgatgtaccgctggtgggcaagtacctcatgtttaccatggtgctagtcaccttctccatcgtcaccagcgtgtgtgtgctcaatgtgcaccaccgctcgcctaccacgcacaccatggccccctgggtcaaggtggtcttcctggagaagctgcccaccctgctcttcctgcagcagccacgccaccgctgtgcacgtcagcgtctgcgcttgaggaggcgccagcgagagcgtgagggcgcaggcgcgcttttcttccgtgaaggtcctgcggctgacccatgtacctgctttgtcaaccccgcctccgtccaaggactcgccggcgcctttagggccgaacctaccgccgctggcccggggcgctctgtggggccatgcagctgtggcctccgggaagcagtggatggcgtacgcttcattgcggaccacatgcgaagtgaggatgatgaccagagtgtgagggaggactggaaatacgttgccatggtgatcgaccgcctgttcctgtggatctttgtctttgtctgtgtctttgggaccgtcggcatgttcctgcagcctctcttccagaactacactgccactaccttcctccaccctgaccactcagctcccagctccaagtga

SEQ ID NO: 29 - DNA, 1502 bp, Rattus norvegicus
tggccgggcactccaactcaatggcgctgttcagcttcagccttctttggctgtgctcaggggttttgggaactgacacagaggagcggctagtggagcatctcttagatccctcccgctataacaagctgattcgtccagctactaacggctctgagctggtgactgtacagctcatggtatcattggctcagctcattagtgtgcacgagcgggagcagatcatgaccaccaatgtctggctgacccaggagtgggaagattaccgcctcacatggaagcctgaggacttcgacaatatgaagaaagtccggctcccttccaaacacatctggctcccagatgtggttctatacaacaatgctgacggcatgtacgaagtctccttctattccaatgctgtggtctcctatgatggcagcatcttttggctaccacctgccatctacaagagtgcatgcaagattgaggtgaagcacttcccatttgaccagcagaattgcaccatgaagtttcgctcatggacctacgaccgtactgagattgacctggtgctcaaaagtgatgtggccagtctggatgacttcacacccagcggggagtgggacatcatcgcactgccaggccgacgcaacgagaacccagacgactccacctatgtggacatcacctatgacttcatcattcgtcgcaaaccactcttctacactatcaacctcatcatcccctgcgtactcatcacctcgctggccatcctggtcttctacctgccctcagactgtggtgaaaagatgacactttgtatttctgtgctgctagcactcacggtgttcctgctgctcatctccaagattatgcctcccacctccctcgatgtaccgctggtgggcaagtacctcatgtttaccatggtgctagtcaccttctccatcgtcaccagcgtgtgtgtgctcaatgtgcaccaccgctcgcctaccacgcacaccatggccccctgggtcaaggtggtcttcctggagaagctgcccaccctgctcttcctgcagcagccacgccaccgctgtgcacgtcagcgtctgcgcttgaggaggcgccagcgagagcgtgagggcgcaggcgcgcttttcttccgtgaaggtcctgcggctgacccatgtacctgctttgtcaaccccgcctccgtccaaggactcgccggcgcctttagggccgaacctaccgccgctggcccggggcgctctgtggggccatgcagctgtggcctccgggaagcagtggatggcgtacgcttcattgcggaccacatgcgaagtgaggatgatgaccagagtgtgagggaggactggaaatacgttgccatggtgatcgaccgcctgttcctgtggatctttgtctttgtctgtgtctttgggaccgtcggcatgttcctgcagcctctcttccagaactacactgccactaccttcctccaccctgaccactcagctcccagctccaagtga

SEQ ID NO: 30 - protein, 629 aa, Rattus norvegicus
MANSGTGAPPPLLLLPLLLLLGTGLLPASSHIETRAHAEERLLKRLFSGYNKWSRPVANISDVVLVRFGLSIAQLIDVDEKNQMMTTNVWVKQEWHDYKLRWDPGDYENVTSIRIPSELIWRPDIVLYNNADGDFAVTHLTKAHLFYDGRVQWTPPAIYKSSCSIDVTFFPFDQQNCTMKFGSWTYDKAKIDLVSMHSRVDQLDFWESGEWVIVDAVGTYNTRKYECCAEIYPDITYAFIIRRLPLFYTINLIIPCLLISCLTVLVFYLPSECGEKVTLCISVLLSLTVFLLLITEIIPSTSLVIPLIGEYLLFTMIFVTLSIVITVFVLNVHHRSPRTHTMPAWVRRVFLDIVPRLLFMKRPSVVKDNCRRLIESMHKMANAPRFWPEPVGEPGILSDICNQGLSPAPTFCNPTDTAVETQPTCRSPPLEVPDLKTSEVEKASPCPSPGSCPPPKSSSGAPMLIKARSLSVQHVPSSQEAAEDGIRCRSRSIQYCVSQDGAASLADSKPTSSPTSLKARPSQLPVSDQASPCKCTCKEPSPVSPVTVLKAGGTKAPPQHLPLSPALTRAVEGVQYIADHLKAEDTDFSVKEDWKYVAMVIDRIFLWMFIIVCLLGTVGLFLPPWLAAC

SEQ ID NO: 31 - protein, 500 aa, Rattus norvegicus
MAGHSNSMALFSFSLLWLCSGVLGTDTEERLVEHLLDPSRYNKLIRPATNGSELVTVQLMVSLAQLISVHEREQIMTTNVWLTQEWEDYRLTWKPEDFDNMKKVRLPSKHIWLPDVVLYNNADGMYEVSFYSNAVVSYDGSIFWLPPAIYKSACKIEVKHFPFDQQNCTMKFRSWTYDRTEIDLVLKSDVASLDDFTPSGEWDIIALPGRRNENPDDSTYVDITYDFIIRRKPLFYTINLIIPCVLITSLAILVFYLPSDCGEKMTLCISVLLALTVFLLLISKIVPPTSLDVPLVGKYLMFTMVLVTFSIVTSVCVLNVHHRSPTTHTMAPWVKVVFLEKLPTLLFLQQPRHRCARQRLRLRRRQREREGAGALFFREGPAADPCTCFVNPASVQGLAGAFRAEPTAAGPGRSVGPCSCGLREAVDGVRFIADHMRSEDDDQSVREDWKYVAMVIDRLFLWIFVFVCVFGTVGMFLQPLFQNYTATTFLHPDHSAPSSK